(19) United States
(12) Patent Application Publication
(10) Pub. No.: US 2024/0270838 A1
(43) Pub. Date: Aug. 15, 2024

(54) METHOD FOR PROPHYLACTIC THERAPY OF CYTOKINE RELEASE SYNDROME AND/OR IMMUNE EFFECTOR CELL-ASSOCIATED NEUROTOXICITY SYNDROME (ICANS)

(71) Applicant: Recordati Rare Diseases Inc., Bridgewater, NJ (US)

(72) Inventor: Timothy Joseph VOORHEES, Burlington, MA (US)

(73) Assignee: Recordati Rare Diseases Inc., Bridgewater, NJ (US)

(21) Appl. No.: 18/436,517

(22) Filed: Feb. 8, 2024

Related U.S. Application Data
(60) Provisional application No. 63/484,370, filed on Feb. 10, 2023.

(51) Int. Cl. C07K 16/24; A61K 39/00; A61K 39/395; A61P 37/06; C07K 16/28

(52) U.S. Cl. CPC C07K 16/248; A61K 39/3955; A61K 39/4611; A61K 39/4631; A61K 39/4643; A61P 37/06; C07K 16/2809; C07K 16/2866; C07K 16/2887; A61K 2039/507; A61K 2039/545; C07K 2317/31; C07K 2317/76

(57) ABSTRACT

A method of prophylactic therapy of a patient who is at risk of the development of cytokine release syndrome (CRS) and/or immune effector cell-associated neurotoxicity syndrome (ICANS) due to a therapeutic intervention;
the method comprising administering an antibody or fragment which is capable of inhibiting human IL-6 to the patient in an antibody dosage regimen in conjunction with the therapeutic intervention;
wherein the antibody dosage regimen comprises administering a pre-emptive dose of the antibody or fragment before the patient is at risk of the development of CRS and/or ICANS
wherein the therapeutic intervention comprises administration of one or more doses of a therapeutic agent.

### CROSS-REFERENCE TO RELATED APPLICATIONS

This application claims the priority benefit of U.S. provisional application No. 63/484,370, filed Feb. 10, 2023, entitled “METHOD FOR PROPHYLACTIC THERAPY OF CYTOKINE RELEASE SYNDROME AND/OR IMMUNE EFFECTOR CELL-ASSOCIATED NEUROTOXICITY SYNDROME (ICANS),” the entire content of which is incorporated herein by reference.

### REFERENCE TO SEQUENCE LISTING

The sequence listing that is contained in the file named “EPCLP0110US_Sequence Listing.xml”, which is 9 KB (as measured in measured in Microsoft Windows®) and created on Feb. 8, 2024, is filed herewith by electronic submission, and is incorporated by reference herein.

### FIELD OF THE INVENTION

The present invention relates to methods for prophylactic therapy of cytokine release syndrome (CRS) and/or immune effector cell-associated neurotoxicity syndrome (ICANS) for which a patient is at risk of suffering due to a therapeutic intervention, such as a CAR T cell therapy, and compositions for use in such methods.

### BACKGROUND TO THE INVENTION

Immunotherapies, especially immune effector cell (IEC) or T cell engaging therapies are promising therapies against cancer, which typically work by directing T cells of the adaptive immune system to kill tumour cells. The concept of tumor-targeted T cells has come to a reality as a result of genetic modification strategies capable of generating a tumor-targeting T-cell receptor. Chimeric antigen receptors (CAR) are recombinant T cell receptors composed of an extracellular domain that can bind specifically to a target molecule expressed on the surface of tumour cells, a transmembrane domain, and an intracellular domain that provides an activation signal to T cells. Patient T cells engineered to express a CAR (CAR T cells) can be activated upon ligation of the CAR with its target antigen. Bispecific antibodies represent an alternative strategy for activating tumor-targeted T cells by binding tumor cell receptors and recruiting cytotoxic immune cells, as described in Jiabing M et al. (2021) Front. Immunol., Sec. Cancer Immunity and Immunotherapy (12), doi.org/10.3389/fimmu.2021.626616.

Immunotherapies including CAR T cell therapy and bispecific antibody therapy are associated with a risk for cytokine release syndrome (CRS) and immune effector cell-associated neurotoxicity syndrome (ICANS) in the days to weeks following administration of the immunotherapy. CRS is characterized by high fever, hypotension, hypoxia, and/or multiorgan toxicity; whereas ICANS is typically characterized by a toxic encephalopathic state with symptoms of confusion and delirium, and occasionally seizures and cerebral oedema. CRS and ICANS are associated with elevated inflammatory cytokines, of which IL-6 is believed to play a significant role in pathology of CRS, whereas the pathophysiology of ICANS is poorly understood and may be more closely related to IL-1, as reported in Garcia Borrega J, Gödel P, Rüger MA, Onur ÖA, Shimabukuro-Vornhagen A, Kochanek M, Böll B. In the Eye of the Storm: Immune-mediated Toxicities Associated With CAR-T Cell Therapy. HemaSphere, 2019; 3:2. dx.doi.org/10.1097/HS9.0000000000000191.

Intensive patient monitoring and management of toxicities is recommended to minimise morbidity and mortality from CRS and/or ICANS. Typical clinical practice in the US is described in Mahmoudjafari, Z et al. American Society for Blood and Marrow Transplantation Pharmacy Special Interest Group Survey on Chimeric Antigen Receptor T Cell Therapy Administrative, Logistic, and Toxicity Management Practices in the United States. Biol. Blood Marrow Transplant. 2019, 25, 26-33. In particular, the anti-IL6 receptor antagonist antibody tocilizumab is approved by the US FDA for treatment of CRS. Corticosteroids may also be used to manage toxicities and are generally reserved as second-line therapy after tocilizumab for symptoms of CRS. Siltuximab is an anti-IL6 antagonist antibody which may be used as second- or third-line therapy for CRS. However, in the absence of clinical trials supporting the use of siltuximab in treatment of CRS, caution is required in its use, and US institutions consider it should not be used for first-line treatment. For ICANS (also known as CAR-T-cell-related encephalopathy syndrome (CRES)), corticosteroids are preferred as first-line treatment compared to tocilizumab, and it is believed that tocilizumab may actually increase incidence of neurotoxicity, because it can increase systemic levels of IL-6 following administration.

Preventive strategies for CRS and/or ICANS are at an earlier stage in development than therapies, although most US institutions use levetiracetam as seizure prophylaxis. Frederick L. Locke et al. describe preliminary results of prophylactic tocilizumab after axicabtageneciloleucel (axi-cel; KTE-C19) treatment for patients with refractory, aggressive Non-Hodgkin Lymphoma (NHL) in Blood. 2017; 130:1547. Prophylactic levetiracetam was also applied. The incidence of severe CRS was lower than observed without prophylaxis, whereas neurotoxicity incidence was not decreased. Favourable results for pre-emptive tocilizumab therapy on CRS, but not ICANS, are also reported in Kadauke S, Myers R M, Li Y, et al. Risk-Adapted Preemptive Tocilizumab to Prevent Severe Cytokine Release Syndrome After CTL019 for Pediatric B-Cell Acute Lymphoblastic Leukemia: A Prospective Clinical Trial. Journal of Clinical Oncology. 2021; 39(8):920-930; and Caimi P F, Pacheco Sanchez G, Sharma A, et al. Prophylactic Tocilizumab Prior to Anti-CD19 CAR-T Cell Therapy for Non-Hodgkin Lymphoma. Front Immunol. 2021; 12:745320.

There remains a need for effective prophylactic therapies for CRS and/or ICANS associated with a therapeutic intervention.

The listing or discussion of an apparently prior-published document in this specification should not necessarily be taken as an acknowledgement that the document is part of the state of the art or is common general knowledge.

### SUMMARY OF THE INVENTION

An object of the invention is to provide a prophylactic therapy for CRS and/or ICANS in a patient who is at risk of suffering either toxicity due to a therapeutic intervention, such as a CAR T cell therapy, or bispecific antibody therapy. Prophylactic therapy with an IL-6 antagonist antibody may commence before the risk of CRS and/or ICANS and may include or continue with one or more further doses in the event that the patient experiences symptoms.

A first aspect of the invention provides a method of prophylactic therapy of a patient who is at risk of the development of cytokine release syndrome (CRS) and/or immune effector cell-associated neurotoxicity syndrome (ICANS) due to a therapeutic intervention;
the method comprising administering an antibody or fragment which is capable of inhibiting human IL-6 to the patient in an antibody dosage regimen in conjunction with the therapeutic intervention;
wherein the antibody dosage regimen comprises administering a pre-emptive dose of the antibody or fragment before the patient is at risk of the development of CRS and/or ICANS; and
wherein the therapeutic intervention comprises administration of one or more doses of a therapeutic agent.

In a related aspect, the invention provides an antibody or fragment which is capable of inhibiting human IL-6 for use in a method of prophylactic therapy of a patient who is at risk of the development of cytokine release syndrome (CRS) and/or immune effector cell-associated neurotoxicity syndrome (ICANS) due to a therapeutic intervention;
the method comprising administering the antibody or fragment to the patient in an antibody dosage regimen in conjunction with the therapeutic intervention;
wherein the antibody dosage regimen comprises administering a pre-emptive dose of the antibody or fragment before the patient is at risk of the development of CRS and/or ICANS; and
wherein the therapeutic intervention comprises administration of one or more doses of a therapeutic agent.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### DESCRIPTION OF THE FIGURES

The following drawings form part of the present specification and are included to further demonstrate certain aspects of the present disclosure. The disclosure may be better understood by reference to one or more of these drawings in combination with the detailed description of specific embodiments presented herein.

FIG. 1. Schematic of IL-6 signalling and sites of action of IL-6 signalling pathway antagonists.

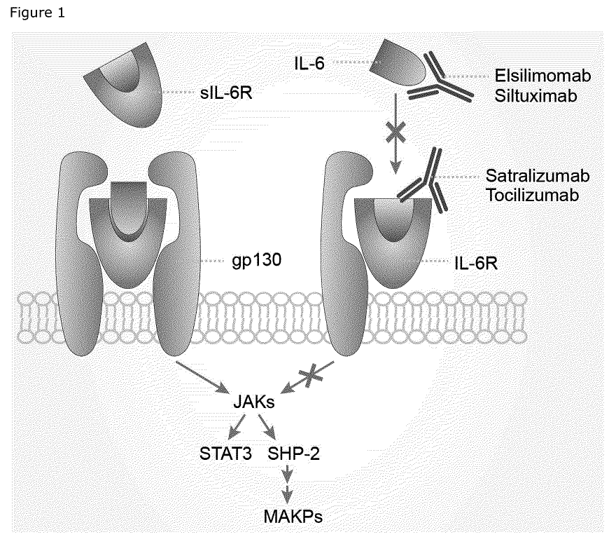

FIG. 2. Clinical trial study design.

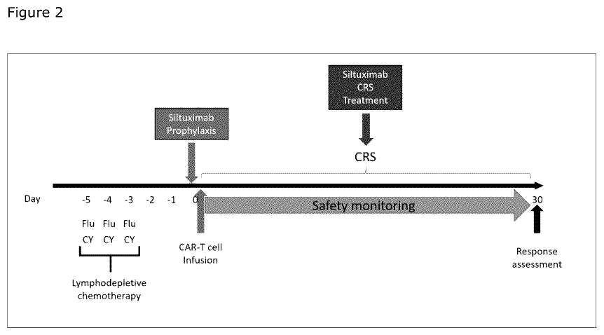

### DETAILED DESCRIPTION OF THE INVENTION

The present invention provides a method for prophylactic therapy of a patient who is at risk of the development of cytokine release syndrome (CRS) and/or immune effector cell-associated neurotoxicity syndrome (ICANS) due to a therapeutic intervention, and compositions for use in the method.

CRS and ICANS are commonly reported toxicities associated with immunotherapies.

Patients who develop CRS are at higher risk of developing ICANS, but patients may develop CRS without ICANS, or ICANS without CRS. ICANS can be biphasic; the first phase may occur concurrently with high fever and other CRS symptoms, typically within the first 5 days after cellular immunotherapy, and the second phase occurs after the fever and other CRS symptoms subside, often beyond 5 days after cell infusion (Neelapu, S., Tummala, S., Kebriaei, P. et al. Chimeric antigen receptor T-cell therapy—assessment and management of toxicities. Nat Rev Clin Oncol 15, 47-62 (2018). doi.org/10.1038/nrclinonc.2017.148). CRS and ICANS may be diagnosed and graded according to the American Society for Transplantation and Cellular Therapy (ASTCT) criteria as provided in Lee D W, Santomasso B D, Locke F L, et al. ASTCT Consensus Grading for Cytokine Release Syndrome and Neurologic Toxicity Associated with Immune Effector Cells. Biology of Blood and Marrow Transplantation. 2019; 25(4): 625-638.3. Grading of CRS and ICANS in adults is described in the Example. ASTCT grading of CRS is based on the presence and/or extent of three classic symptoms, namely fever, hypotension and hypoxia. Alternative criteria for grading CRS may be used, including CTCAE version 4.03, CTCAE version 5.0, Lee criteria, Penn criteria, MSKCC criteria or CARTOX criteria, as described in Lee et al. 2019 supra. ICANS grade in adults is determined by the most severe event selected from five symptom groups (ICE score, level of consciousness, seizure, motor findings, raised ICP/cerebral edema) not attributable to any other cause. A corresponding scoring is used in children, as described in Lee et al. 2019 supra, but CAPD score rather than ICE score is used in children under 12 years old. ICE and CAPD are both encephalopathy assessments. Alternative criteria for grading ICANS may be used, including CTCAE v5.0 or CARTOX criteria, as described in Lee et al. 2019 supra.

By “prophylactic therapy”, the inventors include that the therapy is intended to reduce the risk and/or severity of CRS and/or ICANS in the patient. This may be assessed, for example, by comparing the incidence and/or severity (e.g. grade and/or duration) of CRS and/or ICANS in patients who receive the prophylactic therapy in conjunction with the therapeutic intervention, with patients who receive only the therapeutic intervention (in the context of the same overall treatment scheme). By “prophylactic therapy”, the inventors include the possibility that the patient does develop CRS and/or ICANS and that in such cases, the prophylactic therapy may include therapy intended to reduce the severity of CRS and/or ICANS in the patient.

By “a patient” the inventors intend a human patient. Typically, the patient is an adult i.e. ≥18 years old at the commencement of the method for prophylactic therapy. Alternatively, the patient may be a pediatric patient i.e. <18 years old at the commencement of the method for prophylactic therapy.

### Antibody Dosage Regimen

The method for prophylactic therapy comprises administering an antibody or fragment which is capable of inhibiting human IL-6 to the patient in an antibody dosage regimen in conjunction with the therapeutic intervention. By “antibody dosage regimen in conjunction with the therapeutic intervention” the inventors include that the antibody dosage regimen is applied during the same treatment scheme as the therapeutic intervention. A “treatment scheme” typically includes assessment of a patient as suitable and intended to receive a therapeutic intervention, a therapeutic intervention itself, and a period during which the patient is monitored for signs or symptoms of toxicity i.e. CRS and/or ICANS. The therapeutic intervention comprises administration of one or more doses of one or more therapeutic agents, such as one dose, two doses or three doses. Cell therapies, for example are typically provided as a single dose, but may be given as multiple doses. Bispecific antibody therapies may be provided in multiple doses, which may include an initial treatment cycle comprising one or more smaller dosages before a full dosage, in the form of a step-up dosing schedule; and may comprise multiple treatment cycles. A “treatment scheme” may include pre-conditioning to prepare the patient for the therapeutic intervention, typically provided as one or more doses of a pre-conditioning agent such as a lymphodepletive chemotherapy agent. In instances, the pre-conditioning may comprise a surgical procedure and/or other therapeutic procedure such as radiotherapy. The patient may be monitored before, during and/or after commencement of the therapeutic intervention, or the administration of one or more, and typically all of the doses of the one or more therapeutic agents. Safety monitoring may typically conclude when it is deemed that toxicities associated with the therapeutic intervention have abated to safe levels, and/or that the patient is no-longer at risk of development of toxicities. It is the therapeutic intervention which is associated with the risk of CRS and/or ICANS but it will be appreciated that other features of the treatment scheme, including any pre-conditioning, may moderate the risk of CRS and/or ICANS. Typically, the therapeutic intervention comprises an administration of one or more doses of an immunotherapy agent. Typically, lymphodepletive chemotherapy is provided in one or more doses as pre-conditioning prior to administration of the one or more doses of the immunotherapy agent, particularly as pre-conditioning for immune effector cell therapy, such as CAR T therapy.

The antibody dosage regimen comprises administering a pre-emptive dose of the antibody or fragment before the patient is at risk of the development of CRS and/or ICANS. Thus, the first (and potentially only) dose of the antibody or fragment is administered during the treatment scheme before the time point at which a patient who is to be treated by the therapeutic intervention may typically be at risk of developing CRS and/or ICANS. The timing of this point may vary between different therapeutic interventions but can be established by monitoring patients who have received the same therapeutic intervention, and any pre-conditioning, as intended according to the invention. Where the therapeutic intervention comprises administering more than one dose of one or more therapeutic agents, the risk of CRS and or ICANS may vary depending on the dose. A pre-emptive dose of the antibody or fragment may be administered timed to reduce risk of CRS and/or ICANS associated with each dose of the therapeutic agent which presents the risk. Thus, multiple pre-emptive doses may be administered in any given treatment scheme. A pre-emptive dose subsequent to the first is to be distinguished from a treatment dose because it is administered timed to reduce risk of CRS and/or ICANS associated with a given dose of the therapeutic agent, rather than to address CRS and/or ICANS caused by the preceding dose of the therapeutic agent.

Suitably, the pre-emptive dose of the antibody or fragment is administered between 5 days before and up to 1 or 2 days after the commencement of the administration of at least one of the one or more doses of the therapeutic agent, optionally the immunotherapy agent, such as between 4 days before, 3 days before, 2 days before or 1 day before and up to 1 day after the commencement of the administration of at least one of the one or more doses of the therapeutic agent, optionally immunotherapy agent. The antibody will remain in the patient's circulation over such a time frame, sufficient for it to perform its desired IL-6 inhibitory effect.

Suitably, the pre-emptive dose of the antibody or fragment is administered between 24 hours before and up to 24 hours after the commencement of the administration of the at least one dose of the therapeutic agent, optionally between 2 hours before and at the same time as the commencement of the administration of the at least one dose of the therapeutic agent, optionally the immunotherapy agent. In an embodiment, the pre-emptive dose is administered one hour before the commencement of the administration of the at least one dose of the therapeutic agent. The antibody or fragment is typically administered by intravenous administration, optionally by infusion, optionally over the course of one hour. Thus, the completion of the administration of the antibody or fragment may be timed to coincide with the commencement of the administration of the at least one dose of the therapeutic agent, such as immunotherapy agent.

The antibody or fragment thereof for use of the invention is capable of inhibiting human IL-6. As described in Chen F et al. Measuring IL-6 and sIL-6R in serum from patients treated with tocilizumab and/or siltuximab following CAR T cell therapy. J. Immunol. Methods 2016, 434, 1-8, IL-6 exerts its biological functions via two major pathways: classic signaling and trans-signaling pathways. In the classic signaling pathway, IL-6 binds to the IL-6 receptor (IL-6R) on hepatocytes and some leukocytes. The IL-6 IL-6R complex further recruits the ubiquitously expressed membrane-bound or soluble gp130 (sgp130), triggering the dimerization of gp130 and intracellular signaling. In the trans-signaling pathway IL-6 interacts with soluble IL-6R (sIL-6R) to form the IL-6 sIL-6R complex, which can bind to gp130 on any cell and initiate intracellular signaling without a requirement for the stimulated cell to express IL-6R. An antibody which is capable of inhibiting human IL-6 must be capable of specifically binding to human IL-6, and of inhibiting its interaction with sIL-6R or IL-6R, or otherwise preventing gp130 activation. By “capable of specifically binding”, the inventors include the ability of the antibody or antigen-binding fragment to bind at least 10-fold more strongly to the relevant polypeptide, e.g. IL-6, than to any other polypeptide; preferably at least 50-fold more strongly and more preferably at least 100-fold more strongly. Inhibitory antibodies to IL-6 can typically be divided into two groups; and the putative epitopes on the IL-6 molecule designated Site I and Site II. Site I binders prevent binding to the IL-6R or sIL-6R and thereby prevent gp130 activation. The Site I epitope was further characterized as comprising regions of both amino terminal and carboxy terminal portions of the IL-6 molecule. Site II-binders prevent gp130 activation and therefore may recognize a conformational epitope involved in signalling. Binding of the antibody may be measured by surface plasmon resonance, for example, by immobilizing the antibody on a chip and using recombinant human IL-6 as analyte, as described in WO 2004/039826A1. Suitable antibodies may bind IL-6 with an affinity (Kd) of at least 10-9 M, preferably at least 10-10 M, preferably at least 10-11 or 5×10⁻¹¹ M. Epitope mapping to identify Site I or Site II binders may be performed by binding to human IL-6-mutant proteins as described in Brakenhoff, J. et al. (1990) J. Immunology 145: 561-568). Inhibition of IL-6 activity may be measured by assaying proliferation of the murine B myeloma cell line, 7TD1, in response to IL-6, as described in WO 2004/039826A1. Suitable antibodies may inhibit >50%, such as >90%, such as substantially 100% of 7TD1 cell proliferation in response to IL-6.

By “IL-6” the inventors include any natural or synthetic protein with structural and/or functional identity to the human IL-6 protein, such as defined in UniProt Accession No. P05231, or natural variants thereof. IL-6 gene and/or amino acid sequences are disclosed in Eur. J. Biochem (1987) 168, 543-550; J. Immunol. (1988)140, 1534-1541; and Agr. Biol. Chem. (1990)54, 2685-2688.

By “antibody” the inventors include substantially intact antibody molecules, as well as chimaeric antibodies, humanised antibodies, human antibodies (wherein at least one amino acid is mutated relative to the naturally occurring human antibodies), single chain antibodies, bi-specific antibodies, antibody heavy chains, antibody light chains, homodimers and heterodimers of antibody heavy and/or light chains, and antigen binding fragments and derivatives of the same. The term also includes antibody-like molecules which may be produced using phage-display techniques or other random selection techniques for molecules. The term also includes all classes of antibodies, including IgG, IgA, IgM, IgD, and IgE. Also included for use in the invention are antibody fragments such as Fab, F(ab′)2, Fv, Fab′, scFv (single-chain variable fragment), or di-scFv and other fragments thereof that retain the antigen-binding site. Similarly, the term “antibody” includes genetically engineered derivatives of antibodies such as single-chain Fv molecules (scFv) and single-domain antibodies (dAbs).

Preferred antibodies are chimaeric, such as mouse-human chimaeric antibodies, CDR-grafted antibodies, humanised antibodies, or human antibodies. Although the antibody may be a polyclonal antibody, it is preferred if it is a monoclonal antibody, or that the antigen-binding fragment is derived from a monoclonal antibody. Suitable monoclonal antibodies may be prepared by known techniques, for example those disclosed in “Monoclonal Antibodies; A manual of techniques”, H Zola (CRC Press, 1988) and in “Monoclonal Hybridoma Antibodies: Techniques and Application”, SGR Hurrell (CRC Press, 1982). The antibodies may be human antibodies in the sense that they have the amino acid sequence of human antibodies with specificity for the IL-6; however, it will be appreciated that they may be prepared using methods known in the art that do not require immunisation of humans. Suitable antibodies may be prepared from transgenic mice which contain human immunoglobulin loci, as described in Lee, E., Liang, Q., Ali, H. et al. Complete humanization of the mouse immunoglobulin loci enables efficient therapeutic antibody discovery. Nat Biotechnol 32, 356-363 (2014). doi.org/10.1038/nbt.2825.

Suitably prepared non-human antibodies can be “humanised” in known ways, for example, by inserting the CDR regions of mouse antibodies into the framework of human antibodies. Chimeric antibodies are discussed by Neuberger et al. (1998, 8th International Biotechnology Symposium Part 2, 792-799).

It will be appreciated by persons skilled in the art that the binding specificity of an antibody or antigen-binding fragment thereof is conferred by the presence of complementarity determining regions (CDRs) within the variable regions of the constituent heavy and light chains. As discussed below, in a particularly preferred embodiment of the antibodies and antigen-binding fragments, binding specificity for IL-6 is conferred by the presence of one or more and typically all six of the CDR amino acid sequences defined herein.

Preferably, the antibody or antigen-binding fragment comprises an antibody Fc region. It will be appreciated by the skilled person that the Fc portion may be from an IgG antibody, or from a different class of antibody (such as IgM, IgA, IgD, or IgE). For example, the Fc region may be from an IgG1, IgG2, IgG3, or IgG4 antibody. Advantageously, however, the Fc region is from an IgG1 antibody. It is preferred that the antibody or antigen-binding fragment is an IgG molecule or is an antigen-binding fragment or variant of an IgG molecule.

Suitable antibodies and fragments are described in WO 2004/039826A1. Suitably, the antibody or fragment is a chimeric, humanized or CDR grafted antibody or fragment thereof comprising a heavy chain variable region in which CDR1, CDR2, and CDR3 comprise the amino acid sequences SEQ ID NO: 1, SEQ ID NO: 2 and SEQ ID NO: 3, respectively; and a light-chain variable region in which CDR1, CDR2, and CDR3 comprise the amino acid sequences SEQ ID NO: 4, SEQ ID NO:, 5 and SEQ ID NO: 6, respectively, and a constant region derived from a human IgG antibody.

VH CDR1
(SEQ ID NO. 1)
Ser Phe Ala Met Ser
VH CDR2
(SEQ ID NO. 2)
Glu Ile Ser Ser Gly Gly Ser Tyr Thr Tyr Tyr Pro
Asp Thr Val Thr Gly
VH CDR3
(SEQ ID NO. 3)
Gly Leu Trp Gly Tyr Tyr Ala Leu Asp Tyr
VL CDR1
(SEQ ID NO. 4)
Ser Ala Ser Ser Ser Val Ser Tyr Met Tyr
VL CDR2
(SEQ ID NO. 5)
Asp Thr Ser Asn Leu Ala Ser
VL CDR3
(SEQ ID NO. 6)
Gln Gln Trp Ser Gly Tyr Pro Tyr Thr

In a preferred embodiment the antibody is siltuximab, or an antigen-binding fragment thereof. Siltuximab, also known as CNTO328 and CLLB8, with the US FDA UNII Identifier T4H8FMA7IM and the WHO ATC code L04AC11 is a chimeric (human-murine) IgG1κ monoclonal antibody that binds to human IL-6. The intact molecule contains 1324 amino acid residues and is composed of two identical heavy chains (approximately 50 kDa each) and two identical light chains (approximately 24 kDa each) linked by inter-chain disulfide bonds. Siltuximab contains the antigen-binding variable region of the murine antibody, CLB-IL-6-8, and the constant region of a human IgG1K immunoglobulin.

The complete amino acid sequences of the heavy and light chains of siltuximab are shown below.

Siltuximab heavy chain amino acid sequence
SEQ ID NO. 7
EVQLVESGGKLLKPGGSLKLSCAASGFTFSSFAMSWFRQSPEKRLEWVA
EISSGGSYTYYPDTVTGRFTISRDNAKNTLYLEMSSLRSEDTAMYYCAR
GLWGYYALDYWGQGTSVTVSSASTKGPSVFPLAPSSKSTSGGTAALGCL
VKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQK
SLSLSPGK
Siltuximab light chain amino acid sequence
SEQ ID NO. 8
QIVLIQSPAIMSASPGEKVTMTCSASSSVSYMYWYQQKPGSSPRLLIYD
TSNLASGVPVRFSGSGSGTSYSLTISRMEAEDAATYYCQQWSGYPYTFG
GGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVT
HQGLSSPVTKSFNRGEC

Siltuximab and methods of preparing it, including by recombinant expression of encoding nucleic acid sequences, are described in WO 2004/039826A1.

Other suitable antibodies include olokizumab, which is a IgG4K antibody humanized from rat, and is described in Shaw, S., Bourne, T., Meier, C., Carrington, B., Gelinas, R., & Henry, A., et al. (2014). Discovery and characterization of olokizumab. mAbs, 6(3), 773-781; elsilimomab (also known as B-E8), which is a mouse IgG1K monoclonal antibody described in Wijdenes J, Clement C, Klein B, et al. Human recombinant dimeric IL-6 binds to its receptor as detected by anti-IL-6 monoclonal antibodies. Mol Immunol. 1991; 28(11):1183-1192; or the human monoclonal antibody clone 1339 derived from elsilimomab as described in Fulciniti, M., Hideshima, T., Vermot-Desroches, C., Pozzi, S., Nanjappa, P., Shen, Z., & Tai, Y. T. (2009). A high-affinity fully human anti-IL-6 mAb, 1339, for the treatment of multiple myeloma. Clinical Cancer Research, 15(23), 7144-7152. Further suitable antibodies include clazakizumab (formerly ALD518 and BMS-945429), which is an aglycosylated, humanized rabbit IgG1 monoclonal antibody against interleukin-6, described in Mease P J, Gottlieb A B, et al. (September 2016). “The efficacy and safety of clazakizumab, an anti-interleukin-6 monoclonal antibody, in a phase IIb study of adults with active psoriatic arthritis”. Arthritis Rheumatol. 68 (9): 2163-73; sirukumab, which is a human monoclonal IgG1 kappa antibody described in Smolen J S, Weinblatt M E, Sheng S, Zhuang Y, Hsu B. Sirukumab, a human anti-interleukin-6 monoclonal antibody: a randomised, 2-part (proof-of-concept and dose-finding), phase II study in patients with active rheumatoid arthritis despite methotrexate therapy. Ann Rheum Dis. 2014 September; 73(9):1616-25. doi: 10.1136/annrheumdis-2013-205137. Epub 2014 Apr. 3. PMID: 24699939; PMCID: PMC4145446. Further suitable antibodies include the MH166 antibody (Matsuda, T. et al., Eur. J. Immunol. (1988) 18, 951-956) and the SK2 antibody (Sato, K. et al., The abstracts of the 21st Annual Meeting of the Japanese Society for Immunology (1991) 21, 166). Fragments of any of these antibodies may also be used.

The antibody or fragment should be prepared under sterile conditions. The appropriate volume of antibody or fragment should be withdrawn from the vials. It is recommended that the antibody solution is filtered (0.2 to 1.2 μm) before injection into the patient either by using an in-line filter during infusion or by filtering the solution with a particle filter (e.g., filter Nr. MF1830, Impromediform, Germany). The volume of the antibody is typically added to an infusion bag containing 5% dextrose. Siltuximab is available as a single-use vial containing 100 mg or 400 mg siltuximab powder for concentrate for solution for infusion and should be stored at refrigeration temperature. The siltuximab powder is typically provided with one or more excipients, typically histidine, histidine hydrochloride monohydrate, polysorbate 80, and sucrose. After reconstitution with single-use sterile water for injection, the solution contains 20 mg siltuximab per mL. Antibodies or fragments may be formulated in other ways, as known in the art.

The pre-emptive dose of the antibody when administered by intravenous administration, such as by infusion, may be 11±3 mg/kg patient body weight, optionally 11 mg/kg. A suitable pre-emptive dose of the fragment is a dose having an equivalent antagonistic effect on human IL-6.

The dose of the antibody or fragment is determined according to the weight in kg of the patient. An antibody fragment is to be administered at an equivalent fragment dose having an equivalent antagonistic effect on human IL-6 to the whole antibody from which the fragment is derived. The equivalent fragment dose may be calculated according to the fragment molecular weight compared to the molecular weight of the whole antibody, also referred to as parent antibody. For example, if a given antibody has a molecular weight of 150 kD, and a Fab fragment has a molecular weight of 50 kD, then a fragment dose that is one third of the antibody dose should provide an equivalent antagonistic effect on human IL-6. Thus, if the antibody dose was 12 mg/kg, then the equivalent fragment dose for the Fab fragment would be 4 mg/kg. The equivalent antagonistic effect on human IL-6 may also be determined according to the amount of human IL-6 that the fragment can specifically bind to, compared to the amount of human IL-6 that the parent antibody can specifically bind to. These amounts may be determined by various assays, including ELISA.

Administering the pre-emptive dose of the antibody or fragment may reduce the risk and/or severity of CRS and/or ICANS in the patient. This may be assessed, for example, by comparing the incidence and/or severity (e.g. grade and/or duration) of CRS and/or ICANS in patients who receive the pre-emptive dose in conjunction with the therapeutic intervention, with patients who receive only the therapeutic intervention (in the context of the same overall treatment scheme). Alternatively, a beneficial effect of the pre-emptive dose may be recognised by comparing the incidence and/or severity of CRS and/or ICANS in patients who receive the pre-emptive dose and one or more treatment doses in conjunction with the therapeutic intervention, with patients who receive only the one or more treatment doses in conjunction with the therapeutic intervention (in the context of the same overall treatment scheme).

Suitably, administering the pre-emptive dose of the antibody or fragment reduces the risk that the patient will develop CRS and/or ICANS, such as ≥grade 2 CRS and/or ICANS, such as ≥grade 3 CRS and/or ICANS; and/or reduces the grade of CRS and/or ICANS that the patient is at risk of developing; and/or reduces the duration of CRS and/or ICANS that the patient is at risk of developing.

Typically, the antibody or fragment thereof is the only active agent administered pre-emptively as prophylaxis for CRS and/or ICANS. Alternatively, the pre-emptive dose of the antibody or fragment may be administered in combination with one or more further active agents for prophylaxis of CRS and/or ICANS. By “in combination with” the inventors include that the one or more further active agents are administered between 5 days before and 1 day after commencement of the administration of at least one of the one or more doses of the therapeutic agent administered as the therapeutic intervention. Typically, the administration of the one or more further active agents would commence at the same time as the pre-emptive dose of the antibody or fragment. For example, premedication with a steroid or Obinutuzumab may be provided to decrease risk of CRS, such as where the therapeutic agent is a bispecific antibody. However, the one or more further active agents should be distinguished from those used to decrease acute infusion reactions, such as to immune effector cell therapy, for example, for which premedication with antihistamines and acetaminophen is typical.

As noted above, the prophylactic therapy may comprise one or more treatment doses, subsequent to the pre-emptive dose. Suitably, the antibody dosage regimen comprises administering a first treatment dose of the antibody or fragment after the pre-emptive dose, if clinically indicated. Suitably, the first treatment dose of the antibody or fragment is clinically indicated if the patient develops CRS, optionally if the patient develops ≥grade 1 CRS, optionally if the patient develops ≥grade 2 CRS. Suitably, the first treatment dose of the antibody or fragment is clinically indicated if the patient develops ICANS, optionally if the patient develops ≥grade 1 ICANS, optionally if the patient develops ≥grade 1 ICANS lasting for more than 12 hours. The patient will typically be monitored for toxicity of the antibody or fragment. If the patient experiences dose-limiting toxicity (DLT), defined as unacceptable Grade≥3 treatment-related toxicity or Grade≥3 allergic/hypersensitivity reaction per NCI CTCAE version 5.0 following the pre-emptive dose, treatment with the antibody or fragment is discontinued.

Suitably, the first treatment dose of the antibody or fragment is administered within an hour of diagnosis of CRS and/or ICANS. This is particularly suitable where CRS is diagnosed with or without ICANS, or ICANS is diagnosed in combination with CRS. Alternatively, the first treatment dose of the antibody or fragment may be administered within 24 hours of diagnosis of ICANS, and optionally after ICANS has not improved for at least 6 hours, such as 12 hours. This option is particularly suitable where a further active agent is administered initially, such as within an hour of diagnosis of CRS, and the patient is monitored for improvement for a period of time. Conventionally, a corticosteroid is administered as initial therapy for ICANS.

Suitably, administering the first treatment dose of the antibody or fragment reduces the grade and/or duration of CRS and/or ICANS of the patient, improves overall survival following diagnosis of CRS and/or ICANS, reduces treatment relative mortality at 30 days, reduces number of days of intensive care treatment following diagnosis of CRS and/or ICANS, and/or reduces number of days of inpatient hospital treatment following diagnosis of CRS and/or ICANS. This may be assessed, for example, by comparing the incidence and/or severity (e.g. grade and/or duration) of CRS and/or ICANS, and/or treatment relative mortality at 30 days and/or overall survival, and/or number of intensive care treatment days and/or number of inpatient hospital treatment days in patients who receive the pre-emptive dose and first treatment dose in conjunction with the therapeutic intervention, with patients who receive only the pre-emptive dose in conjunction with the therapeutic intervention (in the context of the same overall treatment scheme). CRS and/or ICANS can be fatal and in such cases death typically follows within 30 days of its diagnosis. Therefore, improvement in patient overall survival would typically be assessed at 30 days from diagnosis. The number of days intensive care treatment or inpatient hospital treatment needed for CRS and/or ICANS may vary depending on the grade of CRS and/or ICANS. Hospital or intensive care treatment days may vary from about 2 days to several weeks, with a median of 4 or 5 days.

The antibody dosage regimen may comprise administering a second treatment dose of the antibody or fragment after the first treatment dose, if clinically indicated. Suitably, the second treatment dose of the antibody or fragment is clinically indicated if CRS and/or ICANS remains at the same grade or greater at 12 hours after the first treatment dose. Suitably, administering the second treatment dose of the antibody or fragment reduces the grade and/or duration of CRS and/or ICANS of the patient and/or improves overall survival following administering the first treatment dose, and/or reduces number of days of intensive care treatment following administering the first treatment dose, and/or reduces number of days of inpatient hospital treatment following administering the first treatment dose. This may be assessed, for example, by comparing the incidence and/or severity (e.g. grade and/or duration) of CRS and/or ICANS and/or overall survival, and/or number of intensive care treatment days and/or number of inpatient hospital treatment days in patients who receive the pre-emptive dose, the first treatment dose and the second treatment dose in conjunction with the therapeutic intervention, with patients who receive only the pre-emptive dose and first treatment dose in conjunction with the therapeutic intervention (in the context of the same overall treatment scheme).

Suitably, the or each treatment dose of the antibody is 11±3 mg/kg patient body weight, optionally 11 mg/kg; or the or each treatment dose of the fragment is a dose having an equivalent antagonistic effect on human IL-6.

The patient may additionally be administered one or more other therapeutic agents for the treatment of CRS and/or ICANS if the patient develops either toxicity. Alternatively, the antibody or fragment provided in the treatment dose(s) may be the sole therapeutic agent administered to the patient to treat CRS and/or ICANS. Suitably, where an additional therapeutic agent is used for the treatment of CRS and/or ICANS, the patient is administered a corticosteroid. Treatment with a corticosteroid may be contemplated in combination with the first treatment dose of the antibody or fragment if the patient develops CRS≥grade 1, or CRS≥grade 2. Treatment with a corticosteroid may be contemplated in combination with the first treatment dose of antibody or fragment if the patient develops ICANS≥grade 1, or ICANS≥grade 1 lasting for more than 12 hours, or ICANS≥grade 2. Treatment with a corticosteroid may be contemplated in combination with the second treatment dose of the antibody or fragment if CRS and/or ICANS remains at the same grade or greater at 12 hours after the first treatment dose. By administering a corticosteroid “in combination with” a treatment dose of antibody or fragment, the inventors include that the corticosteroid and antibody or fragment are administered sufficiently close in time to be active in treatment of CRS and/or ICANS at the same time. This may typically be achieved by commencing the corticosteroid treatment before (e.g. in case of ICANS) or at the same time as the antibody or fragment treatment. Suitable corticosteroids include dexamethasone and methylprednisolone and prednisone. Dexamethasone is suitably administered intravenously at a dose of 10 mg every 6-12 hours. Methylprednisolone (Solumedrol) may be administered intravenously for treatment of grade 4 CRS and/or ICANS, typically at a high dose selected by the physician for the individual patient, such as equivalent to 1000 mg of prednisone.

### Therapeutic Interventions

Immunotherapy is the treatment of disease by eliciting or amplifying an immune response. Two classes or immunotherapy which may present a risk of CRS and/or ICANS are immune effector cell (IEC) therapy and T-cell engaging (TCE) therapy. Thus, in a preferred embodiment, the immunotherapy is an IEC or TCE therapy. In addition, other forms of cell therapy, such as stem cell transplant, may present a risk of CRS and/or ICANS.

An immune effector cells (IEC) may be defined as “a cell that has differentiated into a form capable of modulating or effecting a specific immune response”. IECs represent a relatively new treatment modality. In general, they may be either syngeneic (i.e. derived from the patient to be treated to avoid donor-recipient incompatibility in HLA molecules) or allogeneic (i.e. not derived from the patient to be treated and either engineered to or naturally compatible with genetically non-identical recipients). IEC currently include, but are not limited to, natural killer cells with or without ex vivo activation to exert broad cytotoxicity against tumor cells (NK), genetically-modified T cells expressing CARs or engineered T cell receptors directed against tumor-associated antigens (CAR T or TCR T), cytotoxic T lymphocytes expanded ex vivo against viral or tumor peptides to target infection or malignancy (CTLs), regulatory T cells with or without genetic modification to induce tolerance, dendritic cells loaded with peptides or genetically engineered to express cytokines/chemokines in order to enhance immune recognition (Maus, M. V., Nikiforow, S. The why, what, and how of the new FACT standards for immune effector cells. j. immunotherapy cancer 5, 36 (2017). doi.org/10.1186/s40425-017-0239-0), and tumor infiltrating lymphocytes (TIL), which are a type of CTL. IEC include cytokine-induced killer (CIK) cells, which are a heterogeneous subset of ex-vivo-expanded T lymphocytes that exhibit phenotypical and functional properties of both natural killer (NK) and T cells, predominantly CD3+CD8+ T cells (Sangiolo D. Cytokine induced killer cells as promising immunotherapy for solid tumors. J Cancer 2011; 2:363-8). Between 1993 and 2020 there were 938 registrations for clinical trials if IEC in oncology, including CAR T (51%), NK (15%), TCR T (8%), TIL (8%), and CIK (3%) Jose Vicente Forero-Forero et al. Journal of Clinical Oncology 2021 39:15 suppl, e14516-e14516. Allogeneic cell and NK registrations were increasing rapidly and considered among the most promising IECs. A rising proportion of clinical trial in solid tumors are using CIK and TIL rather than CAR T-cells. Any or all of these classes of IEC may present a risk of CRS and/or ICANS. Currently, the most likely types to present a risk of CRS and/or ICANS are CAR T and TCR T.

Cell therapy agents, including IEC, are typically administered by intravenous infusion, and in one or more dosages in any given treatment scheme. As examples, CAR T therapy and stem cell transplants are typically provided as a single dose, whereas NK cell therapy may be administered in multiple doses.

T-cell engagers (TCE) are a class of biologic therapeutics that have in common the ability to concurrently engage a T-cell surface molecule and a tumoral cell antigen. This class partially overlaps with IEC. There are currently four basic types, classified as cell-based versus soluble, and using either antibody or T cell receptor (TCR) moieties to engage antigen, as described generally in Lowe K L et al. Novel TCR-based biologics: mobilising T cells to warm ‘cold’ tumours, Cancer Treatment Reviews, Volume 77, 2019, Pages 35-43, ISSN 0305-7372, doi.org/10.1016/j.ctrv.2019.06.001. Cell-based TCE are typically tumour-specific human T cells that are engineered to express either an antibody-based chimeric antigen receptor (CAR) or an antigen-specific TCR. Typically, the T cells are sourced from the patient (i.e syngeneic) and expanded ex vivo before adoptive transfer back to the patient. Soluble TCE include bispecific antibodies comprising a first binding specificity for a T-cell surface molecule, and a second binding specificity for a tumoral cell antigen. An alternative soluble TCE comprises a soluble TCR fused to a T-cell surface molecule, such as immune-mobilizing monoclonal TCRs against cancer (ImmTAC), which are soluble TCRs stabilised by a disulphide bond and fused to an anti-CD3 scFv (Lowe et al. 2019, supra). All four of these immunotherapies may be associated with a risk of CRS

(Borrega J, et al. In the Eye of the Storm: Immune-mediated Toxicities Associated With CAR-T Cell Therapy. HemaSphere, 2019; 3:2. dx.doi.org/10.1097/HS9.0000000000000191.

Typically, the therapeutic intervention is for the treatment of a cancer. Immunotherapies including IEC and TCE, including CAR T cells are primarily used to treat cancer. The cancer may be a solid tumour or a blood cancer. Alternatively, the disease to be treated may be a rheumatologic disease, optionally systemic lupus erythematosus (SLE), or a disease caused by a T cell-tropic infectious agent, optionally human immunodeficiency virus. For example, HIV targeted CAR-T, and CD19.CAR-T for rheumatologic disease are contemplated.

Blood cancers that may be treated using immunotherapies including CAR T cells include leukaemia, lymphoma, and multiple myeloma. Examples of Leukaemia that may be treated with immunotherapies including CAR T cells include but are not limited to: B-cell acute lymphoblastic leukaemia (ALL); acute myeloid leukaemia (AML; Buddle et al., 2017, Blood, 130(S1):811; Sallman et al., 2018, Haematologica 103(9):e424-426); T-cell acute lymphoblastic leukaemia (ALL) (Pan et al., 2021, J. Clin. Oncol., 39(30): 3340-3351); T cell lymphoblastic leukaemia; and chronic lymphocytic leukaemia. Examples of lymphoma that may be treated with immunotherapies including CAR T cells include but are not limited to: B-cell non-Hodgkin lymphoma (NHL); follicular lymphoma; mantle cell lymphoma (MCL); large B-cell lymphoma; diffused large B-cell lymphoma, Hodgkin lymphoma, optionally relapsed and refractory Hodgkin lymphoma (Ramos et al., 2020, J. Clin. Oncol., 38(32): 3794-3804); marginal zone lymphoma (MZL); T cell lymphoblastic lymphoma; and small lymphocytic lymphoma.

Solid tumours that may be treated using immunotherapies including CAR T cells include but are not limited to examples set out, for instance, in WO 2020/047306 A1: renal cancer; endometrial cancer; urothelial cancer; oesophageal cancer; ovarian cancer; pancreatic cancer; bladder cancer; placenta cancer; breast cancer; prostate cancer; colorectal cancer; kidney cancer; urethral cancer; thyroid cancer; glioma; testicular cancer; and liver cancer. Further solid tumour cancers that may be treated using immunotherapies including CAR T cells include but are not limited to: glioblastoma (Brown et al., 2016, N. Engl. J. Med., 375:2561-2569; Hedge et al., 2016, JCI, 126(8): 3036-3052); colorectal cancer (Hege et al., 2017, J. ImmunoTher. Cancer, 5:22); breast cancer (Wilkie et al., 2012, J. Clin. Immunol., 32:1059-1070); lung cancer; stomach cancer; liver cancer; cervical cancer; nasopharyngeal carcinoma; metastatic cancer; uveal melanoma; and non-small cell lung cancer.

It is envisaged that the prophylactic therapy for CRS and/or ICANS will not negatively impact the efficacy of the therapeutic intervention, particularly the cancer treatment. For example, it has been described that the IL6 receptor antagonist antibody can be used to prevent CRS following administration of CAR T or bispecific antibodies, without affecting ant-tumour activity, in Caimi et al., Frontiers in Immunology, supra; Kaduake et al., 2021, supra; and Kauer J et al. Tocilizumab, but not dexamethasone, prevents CRS without affecting antitumor activity of bispecific antibodies. J Immunother Cancer. 2020 May; 8(1):e000621. doi: 10.1136/jitc-2020-000621. PMID: 32474413; PMCID: PMC7264835. a potential impact on treatment efficacy can be assessed by comparing a cohort of patients treated with the prophylactic therapy in conjunction with the therapeutic intervention with a cohort who have received only the therapeutic intervention, in the context of the same treatment scheme for the same disease. Efficacy of cancer treatment is typically defined by one or more parameters such as objective response (OR), complete response (CR) or partial response (PR) per applicable response criteria. Further parameters may include progression-free survival (PFS), disease progression determined per applicable response criteria, prolonged stable disease (SD) per applicable response criteria, duration of response (DoR), patient-reported outcomes (PROs) and overall survival (OS).

Suitably, the immunotherapy is CAR T. CAR T or TCR T cells are prepared by the followings steps: leukapheresis of patient blood, separation of T cells (such as by magnetic isolation), T cell activation in vitro (such as using CD3 or CD28 agonists), genomic insertion of the CAR or TCR (such as using viral transduction), T cell expansion, T cell harvest and formulation. A general overview of these steps is provided at world-wide-web at miltenyibiotec.com/GB-en/applications/by-cell-type/t-cells/CAR-T-cell-manufacturing.html?gclid=EAIaIQobChMI7JWm3uvv_AIVQuDtCh1ovA6BEAAYASAAE gLopPD_BwE&countryRedirected=1. Several of these steps are typically performed by the manufacturer. Typically, the clinician performs the step of leukapheresis. Further information on engineering CAR T is provided in Rafiq, S., Hackett, C. S. & Brentjens, R. J. Engineering strategies to overcome the current roadblocks in CAR T cell therapy. Nat Rev Clin Oncol 17, 147-167 (2020). doi.org/10.1038/s41571-019-0297-y. The formulated product is then administered to the patient, typically by intravenous infusion, and typically as a single dose in any given treatment scheme. Dosing is according to the marketing authorisation for the authorised product. For example, tisagenlecleucel is administered to adult patients at a dose of 0.6 to 6.0×10⁸ CAR-positive viable T cells.

IEC and TCE including CAR T cells are suitably directed to a tumoral cell antigen, which may be an antibody antigen or a TCR antigen. By “directed” the inventors include that antibody or TCR moieties of the IEC or TCE are capable of specifically binding to the antigen. Antigens which are intended to be engaged by antibody moieties are typically cell surface antigens. Antigens which are intended to be engaged by a TCR may be cell surface or intracellular antigens. Suitably tumoral antigens, including cell surface antigens are selected from CD19, B cell maturation antigen (BCMA), CD20, CD22, CD30, CD138, CD123, NKG2DL, CD5, CD7, CD4 (Sengsayadeth et al., 2022, EJHaem., 3(S1):6-10), KISS1 R; CLDN6; MUC21; MUC16; SLC6A3; QRFPR; GPR119; UPK2; ADAM12; SLC45A3; MS4A12; ALPP; SLC2A14; GS1-259H13.2; ADGRG2; ECEL1; ERVFRD-1; CHRNA2; GP2; PSG9; IL13Ra2; TAG-72; ErbB2; HER2; B7H3; PD-L1; EPCAM; NKG2D; MESO; CD70; SenL-T7; and CD79b. Preferably, the tumoral antigen is CD19 or BCMA, for which preferred authorised CAR T therapies are available.

For example, CD19 directed CAR T cells may suitably be used to treat B-cell ALL, B-cell NHL, follicular lymphoma, MCL, large B-cell lymphoma; marginal zone lymphoma (MZL); chronic lymphocytic leukaemia; small lymphocytic lymphoma. BCMA directed CAR T cells may suitably be used to treat multiple myeloma. Acute myeloid leukaemia may suitably be treated using CD123 directed CAR T cells (Buddle et al., 2017); NKG2DL directed CAR T cells (Sallman et al., 2018). CD7 directed CAR T cells may suitably be used to treat T-cell acute lymphoblastic leukaemia (Pan et al., 2021) and/or T-cell lymphoblastic lymphoma. CD5 directed CAR T cells may be suitably used to treat T-cell acute lymphoblastic leukaemia and/or lymphoma. SenL-T7 directed CAR T cells may suitably be used to treat CD7+ T-cell Lymphoblastic Leukemia or T-cell Lymphoblastic Lymphoma. CD79b directed CAR T cells may suitably be used to treat Acute Lymphoblastic Leukemia and B-cell Non-Hodgkin's Lymphoma. CD22 directed CAR T cells may suitably be used to treat Relapsed/Refractory Leukemia or Lymphoma.

Suitable antigen targets for solid tumor treatment include those of the following table, as listed in Chen, L., Xie, T., Wei, B., & Di, D. (2022). Current progress in CAR-T cell therapy for tumor treatment (Review). Oncology Letters, 24, 358. doi.org/10.3892/ol.2022.13478:

Popular targets in solid tumor treatment

| Tumor site/ | Potential therapeutic |
|---|---|
| origin | target for treatment |
| Skin | GD2, VEGFR |
| Head and neck | EGFR |
| Brain | EGFRvIII, HER2, IL-13RA |
| Nerve cells | GD2, PHOX2B |
| Lungs | CEA, EGFR, HER2, MSLN, |
| | CLDN18, ROR, GD2 |
| Breast | GD2, EGFR, ROR, TEM8, |
| | HER2, MSLN |
| Stomach | CEA, HER2, EpCAM, CLDN18, |
| | MSLN |
| Liver | CEA, GPC3, HER2 |
| Pancreas | CEA, MSLN, MUC1, HER2, |
| | CLDN18, EGFR |
| Ovary | CEA, MSLN, L1CAM, MUC16, |
| | CLDN18, PSCA |
| Kidney | CAIX, VEGFR |
| Prostate | PSMA, PSCA |
| Colon | CEA, GUCY2C, CLDN18 |
| Soft tissue | GD2, HER2 |

GD2, diasialoganglioside 2; VEGFR, vascular endothelial growth factor receptor; EGFR, epidermal growth factor receptor; EGFRVIII, EGFRv variant III; HER2, human epidermal growth factor receptor 2; IL-13RA, interleukin-13RA; PHOX2B, paired-like homebox 2B; MSLN, mesothelin; CLDN18, claudin 18; ROR, tyrosine protein kinase transmembrane receptor; TEM8, tumor endothelial marker 8; CEA, carcinoembryonic antigen; EpCAM, epithelial cell adhesion molecule; GPC3, glycipan 3; MUC1, mucin; L1CAM, L1 cell adhesion molecule; PSCA, prostate stem cell antigen; CAIX, carbonic anhydrase IX; PSMA, prostate-specific membrane antigen; GUCY2C, guanylate cyclase 2C.

In addition, renal cancer may suitably be treated using KISS1 R directed CAR T cells, SLC6A3 directed CAR T cells, CD70 directed CAR T cells. CLDN6 directed CAR T cells may suitably be used to treat endometrial cancer and/or urothelial cancer. MUC16 directed CAR T cells or MESO directed CAR T cells may suitably be used to treat ovarian cancer. MUC21 directed CAR T cells may suitably be used to treat oesophageal cancer. GPR119 directed CAR T cells may suitably be used to treat pancreatic cancer. UPK2 directed CAR T cells may suitably be used to treat urothelial cancer and/or bladder cancer. ADAM12 directed CAR T cells may suitably be used to treat placenta cancer, breast cancer, and/or pancreatic cancer. Prostate cancer may suitably be treated using SLC45A3 directed CAR T cells and/or ACPP directed CAR T cells. MS4A12 directed CAR T cells may suitably be used to treat colorectal cancer. ALPP directed CAR T cells may suitably be used to treat endometrial cancer and/or ovarian cancer. SLC2A14 directed CAR T cells may suitably be used to treat testicular cancer. GS1-259H13.2 directed CAR T cells may suitably be used to treat thyroid cancer, glioma, and/or testicular cancer. ERVFRD-1 directed CAR T cells may suitably be used to treat kidney cancer or urethral cancer. ADGRG2 directed CAR T cells may suitably be used to treat ovarian cancer. ECEL1 directed CAR T cells may suitably be used to treat endometrial cancer. CHRNA2 directed CAR T cells may suitably be used to treat prostate cancer. GP2 directed CAR T cells may suitably be used to treat pancreatic cancer. PSG9 directed CAR T cells may suitably be used to treat kidney cancer or liver cancer (WO 2020/047306 A1). IL13Ra2 directed CAR T cells and/or HER2 directed CAR T cells may suitably be used to treat glioblastoma (Brown et al., 2016; Hedge et al., 2016). TAG-72 directed CAR T cells may suitably be used to treat colorectal cancer (Hege et al., 2017). ErbB2 and/or MUC1 directed CAR T cells may suitably be used to treat breast cancer (Wilkie et al., 2012). MUC1 directed CAR T cells may suitably be used to treat oesophageal cancer. B7H3 directed CAR T cells may suitably be used to treat ovarian cancer. PD-L1 directed CAR T cells may suitably be used to treat lung cancer. EPCAM directed CAR T cells may suitably be used to treat liver cancer, stomach cancer, nasopharyngeal carcinoma, and other solid tumours. NKG2D directed CAR T cells and CEA directed CAR T cells may suitably be used to treat metastatic cancers such as liver metastatic colorectal cancer.

Suitably, the CAR T cells are selected from the group consisting of tisagenlecleucel, axicabtagene ciloleucel, brexucabtagene autoleucel and lisocabtagene maraleucel, relma-cel, idecabtagene vicleucel or ciltacabtagene autoleucel. Suitable CD19 directed CAR T cells may be selected from the group consisting of tisagenlecleucel, axicabtagene ciloleucel, brexucabtagene autoleucel and lisocabtagene maraleucel. This group may also include Relma-cel, which is authorised in China for diffuse large B cell lymphoma (DLBCL). Suitably, the BCMA directed CAR T cell is selected from idecabtagene vicleucel or ciltacabtagene autoleucel. These are currently authorised in the US for indications shown in the table below, reproduced from world-wide-web at cancer.gov/about-cancer/treatment/research/car-t-cells.

US FDA-Approved CAR T-Cell Therapies

| | Brand | Target | Targeted | |
|---|---|---|---|---|
| Generic Name | Name | Antigen | Disease | Patient Population |
| Tisagenlecleucel | Kymriah | CD19 | B-cell acute | Children and young adults |
| | | | lymphoblastic | with refractory or relapsed |
| | | | leukemia (ALL) | B-cell ALL |
| | | | B-cell non-Hodgkin | Adults with relapsed or |
| | | | lymphoma (NHL) | refractory B-cell NHL |
| Axicabtagene | Yescarta | CD19 | B-cell non-Hodgkin | Adults with relapsed or |
| ciloleucel | | | lymphoma (NHL) | refractory B-cell NHL |
| | | | Follicular lymphoma | Adults with relapsed or |
| | | | | refractory follicular lymphoma |
| Brexucabtagene | Tecartus | CD19 | Mantle cell | Adults with relapsed or |
| autoleucel | | | lymphoma (MCL) | refractory MCL |
| | | | B-cell acute | Adults with refractory or |
| | | | lymphoblastic | relapsed B-cell ALL |
| | | | leukemia (ALL) | |
| Lisocabtagene | Breyanzi | CD19 | B-cell non-Hodgkin | Adults with relapsed or |
| maraleucel | | | lymphoma (NHL) | refractory B-cell NHL |
| Idecabtagene | Abecma | BCMA | Multiple myeloma | Adults With relapsed or |
| vicleucel | | | | refractory multiple myeloma |
| Ciltacabtagene | Carvykti | BCMA | Multiple myeloma | Adults with relapsed or |
| autoleucel | | | | refractory multiple myeloma |

Suitably, the immunotherapy agent is a bispecific antibody, particularly a T cell engaging bispecific antibody. Thus, suitable bispecific antibodies comprise a first binding specificity for a T cell surface molecule, and a second binding specificity for a tumoral cell antigen. Suitable T cell surface molecules are CD3, CTLA4, LAG3 or CD16A. Suitable tumoral cell antigens are cell surface tumoral cell antigens. Suitable antigens may be the same as those targeted by CAR T, or alternative targets, including CD19, BCMA, CD20, CD22, CD30, CD79, CD138, PD1, GP100, EpCAM, GPRC5D, CD123, DLL3. Preferred tumoral cell antigens are CD19 and BCMA, for which preferred bispecific antibodies are approved.

Suitably, the bispecific antibody is a full-size IgG-like asymmetric bispecific antibody, optionally selected from the group consisting of Triomab, CrossMab, Duobody and BEAT; or is a single-chain variable fragment (scFv) antibody, optionally selected from the group consisting of bispecific T-cell engager (BiTE), dual-affinity re-targeting protein (DART), Tandem diabody (TandAb) and Immunotherapy antibody (Itab). These formats are described in Wang Q, Chen Y, Park J, Liu X, Hu Y, Wang T, McFarland K, Betenbaugh M J. Design and Production of Bispecific Antibodies. Antibodies (Basel). 2019 Aug. 2; 8(3):43. Doi: 10.3390/antib8030043. PMID: 31544849; PMCID: PMC6783844; and Lowe et al. 2019, supra. Triomab is an IgG format with two antigen-targeting domains formed of variable heavy (VH) and variable light (VL) chains. Duobody is an IgG format with two VH/VL antigen-targeting domains and a silenced Fc domain. CrossMAb is an IgG format with three VH/VL antigen targeting domains and a silenced Fc domain. Bispecific Engagement by Antibodies based on T cell receptor (BEAT) is an IgG format with one VH/VL, one scFv targeting domain and the Fc region silenced and engineered to mimic the TCR. Dual Affinity Re-Targeting (DART) is two VH/VL targeting domains linked and stabilised by a disulphide bond. Bispecific T cell Engager (BITE) is two VH/VL regions engineered as scFvs and connected by flexible linker peptides. TandAb is four VH/VL targeting domains linked. ImmunoTherapy antibody (Itab) is two VH/VL targeting domains linked. The specific linkages are illustrated in Lowe et al. 2019, supra.

T-cell engaging bispecific antibodies which have been approved for use

| | | | First Approved | |
|---|---|---|---|---|
| Drug Name | Company | Targets | Date (Country) | Indications |
| Blinatumomab | Amgen | CD3/ | December 2014 | Relapsed or refractory |
| | | CD19 | (USA) | precursor B-cell acute |
| | | | | lymphoblastic leukemia(ALL) |
| Tebentafusptebn | Immunocore | GP100/ | January 2022 | unresectable or metastatic |
| | | CD3 | (USA) | uveal melanoma |
| Cadonilimab | Akeso, Inc. | PD-1/ | June 2022 | cervical cancer |
| | | CTLA-4 | (China) | |
| Mosunetuzumab | Roche | CD20/ | June 2022 | relapsed or refractory (R/R) |
| | | CD3 | (EU) | follicular lymphoma (FL) |
| Teclistamab | Janssen | BCMA/ | August 2022 | relapsed and refractory |
| | | CD3 | (EU) | multiple myeloma |

Source: world-wide-web at biochempeg.com/article/252.html

T-cell engaging bispecific antibodies in development

| | | | |
|---|---|---|---|
| Drug | Company | Targets | Key indications |

Phase III trials

| | | | |
|---|---|---|---|
| Glofitamab | Roche/Genentech/ | CD20/CD3 | DLBCL |
| | Chugai | | |
| Epcoritamab | Genmab/Abbvie | CD20/CD3 | DLBCL |
| Elranatamab | Pfizer | BCMA/CD3 | Multiple Myeloma |
| Erfonrilimab | Alphamab | PDL1/CTLA4 | NSCLC, PDAC |
| | Oncology | | |
| Tebotelimab | MacroGenics | PD1/LAG3 | Gastric/GOJ |
| | | | cancer |
| Catumaxomab | LintonPharma | EpCAM/CD3 | Gastric |
| | | | adenocarcinoma |

Early phase development

| | | | |
|---|---|---|---|
| Odronextamab | Regeneron | CD20/CD3 | Non-Hodgkin's |
| | | | lymphoma |
| Talquetamab | Janssen | GPRC5D/CD3 | Multiple myeloma |
| Flotetuzumab | MacroGenics | CD123/CD3 | Acute myeloid |
| | | | leukemia |
| AFM13 | Affimed | CD30/CD16A | Peripheral T-cell |
| | | | lymphoma |
| Tarlatamab | Abbvie/Amgen | DLL3/CD3 | Small-cell lung |
| | | | cancer |
| REGN5458 | Regeneron | BCMA/CD3 | Multiple myeloma |
| TNB-383B | Abbvie | BCMA/CD3 | Multiple myeloma |

Source: world-wide-web at biochempeg.com/article/252.html, with reference to Esfandiari et al., (2022).
Bispecific antibodies in oncology.
Nature Reviews Drug Discovery, doi.org/10.1038/d41573-022-00040-2

Suitably, the bispecific antibody is selected from the group consisting of Blinatumomab, Tebentafusp-tebn, Cadonilimab, Mosunetuzumab, Glofitamab, Epcoritamab, Teclistamab, Elranatamab, Erfonrilimab, Tebotelimab, Catumaxomab, Odronextamab, Talquetamab, Flotetuzumab, AFM13, Tarlatamab, TNB-383B, and REGN5458.

Typically, a bispecific antibody is administered multiple times throughout the course of a treatment scheme. Typically, it may be administered in multiple “treatment cycles”. The term “treatment cycle” as used herein means a course of one or more treatments or treatment periods that is repeated on a regular schedule and may encompass a period of rest. For example, a treatment given for four weeks followed by two weeks of rest is one treatment cycle of six weeks. Treatment cycles may be of 2 weeks, 3 weeks, 4 weeks, 5 weeks, 6 weeks, 7 weeks, 8 weeks, 9 weeks, 10 weeks, 11 weeks, 12 weeks etc. The treatment cycle may be repeated, either identically or in an amended form, e.g., with a different dose or schedule, or with different additional treatments. Thus, the treatment scheme may comprise one or more treatment cycles. These may belong to distinct treatment phases i.e. induction, consolidation and maintenance. A treatment scheme may comprise one or more induction treatment cycles. Following the one or more induction treatment cycles, the treatment scheme may comprise one or more consolidation treatment cycles. Following one or more consolidation treatment cycles, the treatment scheme may comprise one or more maintenance treatment cycles. A clinical decision may be taken during or after induction or consolidation treatment cycles as to whether the patient should go on to the next phase. Any or all of these treatment phases, and their constituent treatment cycles, may present a risk of CRS and/or ICANS. For example, in a clinical trial, each treatment cycle in a treatment scheme comprising induction, consolidation and maintenance treatment cycles involving Blinatumomab presented a risk of CRS, which was mitigated by administering dexamethasone (Rambaldi A, Huguet F, Zak P, Cannell P, Tran Q, Franklin J, Topp M S. Blinatumomab consolidation and maintenance therapy in adults with relapsed/refractory B-precursor acute lymphoblastic leukemia. Blood Adv. 2020 Apr. 14; 4(7): 1518-1525. doi: 10.1182/bloodadvances.2019000874. PMID: 32289160; PMCID: PMC7160264). Alternatively, treatment cycles subsequent to the first induction treatment cycle or the induction phase may be associated with a lower or no risk of CRS and/or ICANS. Thus, according to the invention, the pre-emptive dose of the antibody or fragment may be administered timed to reduce risk of CRS and/or ICANS associated with any or all of the treatment cycles, typically at least the initial treatment cycle, and typically by repeat administration at or near the start of each administration of a bispecific antibody which presents a risk of CRS and/or ICANS.

On the initial treatment cycle, a bispecific antibody is typically administered in one, two or three doses, which may create a risk of CRS and/or ICANS. These initial doses may form a step-up dosing schedule at the beginning of a therapeutic intervention. For example, a small dose may be administered as a first dose, an intermediate dose may be administered as a second dose, and a full dose may be administered as a third dose. In such a schedule, the first dose may be administered on day 1, the second dose on day 8 and the third dose on day 15. Typically, the highest risk of CRS and/or ICANS is encountered following the full (e.g. third) dose. Thus, the pre-emptive dose of the antibody or fragment may be administered timed to reduce risk of CRS and/or ICANS associated with the full (e.g. third) dose. Alternatively, it may be administered to reduce risk of CRS and/or ICANS of the or each earlier dose which is not the full dose of a step-up dosing schedule.

Typically, a bispecific antibody is administered by a parenteral route, such as intravenously, optionally by intravenous infusion, or by subcutaneous injection. The dosing schedule, including timing, dosage and route of administration are provided in the marketing approval of authorised bispecific antibodies. Teclistamab, for example, is administered on days 1, 4 and 7 at increasing dosages, specifically at 0.06, 0.3 and then 1.5 mg/kg patient body weight, by subcutaneous injection. The dose at day 7 is the full dose, and weekly dosing commences a week after the full dose as a continued full-dose therapy. According to the marketing approval, one to three hours before receiving each of the doses of the step-up dosing schedule, the patient is administered a corticosteroid to reduce the risk of developing CRS.

Any or all of the features described above in relation to the first aspect of the invention relating to a prophylactic method may be applied in relation to the corresponding aspect of the invention, which provides a composition for use.

Preferences and options for a given aspect, feature, or parameter of the invention should, unless the context dictates otherwise, be regarded as having been disclosed in combination with any and all preferences and options for all other aspects, features, and parameters of the invention.

All documents are incorporated by reference in their entirety.

### EXAMPLES

The present invention will be further illustrated in the following examples, without any limitation thereto.

### Example 1: Phase I Study of Cytokine Release Syndrome and/or Immune Effector Cell-Associated Neurotoxicity Syndrome Prophylaxis and Treatment with Siltuximab

### Background and Rationale

### 1.1 Study Synopsis

OSU-22031 is a single-center phase I study to assess the safety of administering siltuximab prophylaxis prior to standard of care CD19 directed chimeric antigen receptor T-cell therapy (CD19.CAR-T).

### Treatment Plan:

Subjects with CD19 positive non-Hodgkin lymphoma who are planned to undergo standard of care CD19.CAR-T cell therapy will be treated with siltuximab prophylaxis at a dose of 11 mg/kg IV infusion 1 hour prior to CD19.CAR-T infusion. Monitoring for CRS and/or ICANS will be per institutional standards. For any grade 1 ICANS lasting more than 12 hours or any ≥grade 2 CRS, subjects will be eligible to receive a treatment dose of siltuximab 11 mg/kg. If CRS and/or ICANS remains the same grade at 12 hours after first treatment dose, subjects will be eligible to receive a second treatment dose of siltuximab 11 mg/kg. Any worsening of CRS and/or ICANS grade after first treatment dose will be considered siltuximab failure and the subject will receive standard of care tocilizumab and/or steroids per institutional standards.

### Schema:

### Eligibility

Adult≥18 years
CD19 positive non Hodgkin lymphoma receiving commercially approved CD19.CAR-T cells

### Treatment

### Day 0:

Siltuximab 11 mg/kg IV infusion, 1 hour prior to CAR-T cell infusion
AntiCD19 CAR-T cell infusion

### CRS/ICANS Treatment (During Day 0-30): (See Appendix E and F)

Subjects with grade 1 ICANS lasting more than 12 hours or grade≥2 CRS will be treated with a treatment dose of Siltuximab 11 mg/kg IV infusion over 1 hour.
If CRS and/or ICANS is the same grade at 12 hours after first treatment dose, administer a second treatment dose of Siltuximab 11 mg/kg IV infusion over 1 hour.
If CRS and/or ICANS grade worsens at any time after completion of the first treatment dose, will administer rescue tocilizumab dose and/or steroids per institutional standards.

### Disease Assessment

Standard of care CT Chest, Abdomen, Pelvis or PET/CT at ˜30 days and ˜90 days.
Clinical follow up for 1 year.

### Primary Objective

To determine the safety of siltuximab when given prior to CD19.CAR-T cells for prevention of cytokine release syndrome associated CAR-T cell therapy.

### Secondary Objectives

To describe the safety profile of siltuximab as first line treatment of new onset grade≥2 CRS.
To estimate the incidence of all-grade CRS and grade≥3 CRS after CAR-T cell therapy with siltuximab prophylaxis.
To estimate the incidence of all grade ICANS and grade≥3 ICANS after CAR-T cell therapy with siltuximab prophylaxis.
To describe the response rates of grade≥2 CRS to treatment with a second dose of siltuximab.
To describe the disease response rates of lymphoma patients after CD19.CAR-T cell therapy with siltuximab prophylaxis.
To estimate the Progression free survival (PFS) in subjects with treated with CAR-T cell therapy with siltuximab prophylaxis.
To estimate the Overall Survival (OS) in subjects with treated with CAR-T cell therapy with siltuximab prophylaxis.

### Exploratory Objectives

To describe CD19.CAR-T cell expansion and persistence following treatment with siltuximab prophylaxis.
To describe changes in plasma cytokine concentrations in patients treated with CD19.CAR-T cells with prophylactic siltuximab.
To describe changes in peripheral blood immunophenotypes in patients treated with CD19.CAR-T cells with prophylactic siltuximab.
To measure complement activation and evidence of microangiopathic hemolytic anemia following CD19.CAR-T cell therapy after siltuximab prophylaxis.
To examine the role of complement activation on development and persistence of ICANS.
To examine the correlation between apheresis product, infusional CAR-T product, and pre-lymphodepletion immunophenotypes with CRS/ICANS and CD19.CAR-T clinical response.
   1.2 Cytokine Release Syndrome and Neurotoxicity after CD19.CAR-T Cell Therapy
T cells are a component of the adaptive immune system, as effectors of cell-mediated immunity. T cells exert their cytotoxic and potentially anti-tumoral effect upon engagement of the T-cell receptor by a cognate peptide antigen, which is presented in the context of a specific major histocompatibility complex (MHC) molecule. The concept of tumor-targeted T cells has come to a reality as a result of genetic modification strategies capable of generating a tumor-targeting T-cell receptor. Chimeric antigen receptors (CAR) are recombinant T cell receptors composed of an extracellular fragment derived from immunoglobulin variable fragment, as single chain (scFv), which is in turn linked to intracellular signaling sequences that are derived from T cells. Insertion of a CAR in a T cell can induce activation of the T cell upon ligation of the scFv with its target antigen. Adoptive immunotherapy with anti-CD19 CAR-T cells (CD19.CAR-T) has demonstrated activity in the treatment of relapsed or refractory B cell lymphomas.
Treatment with CAR-T cells is associated with well-described acute adverse events, including cytokine release syndrome (CRS) and immune effector cell associated neurologic syndrome (ICANS). In larger CAR-T studies reported in lymphoma, CRS and ICANS occurred in 58 and 21% respectively in lymphoma patients treated with tisagenlecleucel and 93 and 64%, respectively in axicabtagene ciloleucel.^{1,2}
The acute complications of CAR-T cell therapy are the result of rapid CAR-T cell expansion and a hyperinflammatory state related to the activation of the CAR-T cells, in conjunction with other compartments of the immune system, such as monocytes and macrophages.^{3,4} The cytokine responses in CRS and ICANS have interleukin 6 as a central mediator.⁵

### 1.3 Treatment of CRS

Cytokine release syndrome can be successfully treated with tocilizumab, a humanized monoclonal antibody that binds the IL-6R in both its soluble and membrane-bound forms, preventing both “trans” and “classic” IL-6 signaling.⁶ Earlier CAR-T trials in acute lymphoblastic leukemia identified CRS as an immune complication of this therapy that could be treated successfully with tocilizumab.⁷ Subsequent trials of CAR-T cell products in relapsed non-Hodgkin lymphoma reported on the use of tocilizumab for treatment of CRS without affecting disease control or CAR-T cell expansion.^{1,2,8}
The role of tocilizumab in the treatment of CRS has been well established, highlighting the central role of IL-6 signaling in its pathophysiology.⁸ Animal studies have replicated CAR-T cell-associated CRS and neurotoxicity, indicating the relevance of bystander monocytes as sources of key cytokines, including IL-6 and IL-1.4 Clinicopathologic studies conducted on tissues from a patient that died after CRS also suggest that endothelial cells express IL-6.9 Tocilizumab has been observed in animal models and clinical trials to treat CRS, but it does not have therapeutic effect on neurotoxicity. Norelli and colleagues observed that tocilizumab administration to mice prevented CAR-T associated CRS but not neurotoxicity.⁴
Early clinical observations in the treatment of CAR-T associated CRS indicated that tocilizumab could control the fever and hemodynamic instability, only to be followed by neurotoxicity.¹⁰ The mechanisms postulated for the ensuing neurotoxicity included: direct neurologic effect of IL-6, observed in neurodegenerative or systemic inflammatory disorders; elevated IL-6 levels in the CNS; and decreased clearance of IL-6 after tocilizumab blockade of IL-6R, leading to transient increases in IL-6.^{11,12} Subsequent clinical trials of axi-cel and tis-cel confirmed the profile of CAR-T cell mediated CRS, without a reported association between tocilizumab and ICANS.^{1,2}

### 1.4 Preventive Measures for CRS

Investigators from the Children's Hospital of Philadelphia reported the results of a prospective clinical trial of risk-adapted, pre-emptive tocilizumab therapy after tisa-cel in children and young adults with acute lymphoblastic (ALL).¹³ Patient with high tumor burden (defined as 40% or more blasts in bone marrow aspirate)(n=15), were administered tocilizumab (8-12 mg/kg) at the time of persistent fever, defined as two temperature measurements ≥38.5° C. in a 24 hour period. Treatment of CRS for this cohort and for the low tumor burden group were done per institutional standards. All 15 high tumor burden patients received pre-emptive tocilizumab and 4/15 (27%) developed grade 4 CRS at a median time to CRS of 2 days (range 2/7). Two out of 55 (3.6%) low tumor burden patients developed grade 4 CRS. Neurotoxicity was observed in 4 patients, 9 in the high tumor burden (60%) and 10 (18%) in the low tumor burden, with grade 3-4 events observed in 4 patients (2 in each cohort). Comparison with a historical cohort of high tumor burden patients showed lower rates of grade 4 CRS and lower number of additional doses of tocilizumab, with comparable rates of corticosteroid use. There were no differences in the rates of grade≥2 neurologic events (53% vs. 54%) and efficacy was also similar between the two cohorts.
A safety expansion cohort of NHL patients treated in the ZUMA-1 trial were treated with prophylactic levetiracetam on day 0 (750 mg twice daily) and tocilizumab two days after axi-cel infusion.¹⁴ The incidence of severe CRS was lower than observed without prophylaxis, whereas neurotoxicity incidence was not decreased, with a potential increase in grade≥3 events. This decrease in CRS severity observed with day 2 tocilizumab infusion after axi-cel is compatible with our observations listed below, whereas the persistence in neurotoxicity may be secondary to differences in the toxicity profile of CD3ζ-CD28 vs. CD3ζ-41BB CAR-T products and may be related to increases in IL-6 with axi-cel. In addition, the later timing of the prophylaxis strategy reported by Locke and colleagues may also have affected the incidence of adverse events, since significant cytokine changes can be observed as early as day 2. Earlier and full IL-6 axis blockade, as proposed in our study, may further prevent escalation of the cytokine storm, impeding reaching a cytokine milieu that leads to a self-sustaining inflammatory state.

### 1.4.1 Tocilizumab Prophylaxis for CRS, Experience at the Case Comprehensive Cancer Center

Co-investigators from the Case Comprehensive Cancer Center have investigated the use of tocilizumab as prophylactic agent for prevention of severe CRS in adult non-Hodgkin lymphoma patients treated with anti-CD19 CAR-T cells. In May of 2019, University Hospitals Seidman Cancer Center instituted a protocol for administration of tocilizumab at a dose of 8 mg intravenously, 1 hour prior to CAR-T infusion. Twenty patients were treated with prophylactic tocilizumab and 8 subjects without tocilizumab. Cell products included locally manufactured anti-CD19 CAR-T (n=18) and tisagenlecleucel (n=2). There were no adverse events attributed to tocilizumab. Ten patients had grade 1-2 CRS at a median of 4 (range 3-7) days. There were no cases of grade≥3 CRS. Five patients had ICANS, grade 1 (n=4) and grade 4 (n=1).15

### 1.5 Siltuximab

### 1.5.1 Overview

Siltuximab (previously referred to as CNTO 328) is a first-in-class chimeric (human-mouse) immunoglobulin G1κ (IgG1κ) monoclonal antibody (mAb) against human interleukin (IL)-6.

### 1.5.2 Safety

Adverse drug reactions (ADRs) were reported in 87 Multicentric Castleman's Disease (MCD) patients treated with siltuximab at the recommended dosage of 11 mg/kg q3 weeks. The most frequent ADRs (in >20% of patients) during treatment with siltuximab in the MCD clinical trials were upper respiratory tract infection, pruritus, rash, arthralgia, and diarrhea. The most serious ADR associated with the use of siltuximab was anaphylactic reaction.

### 1.5.3 Clinical Trial CNTO328MCD2001

This was a randomized, double-blind, placebo-controlled, multicenter, Phase 2 study to determine the safety and efficacy of siltuximab+best supportive care compared with best supportive care, in patients with symptomatic MCD. Study CNTO328MCD2001 met its hypothesis on the primary efficacy endpoint by showing a statistically significant improvement in independently reviewed durable tumor and symptomatic response rate in the siltuximab group compared with the placebo group (34% vs 0%, respectively; 95% CI of the difference: 11.1, 54.8; p=0.0012).¹⁶

### 1.6 Rationale of Siltuximab for Preventing CRS and ICANS

The importance of IL-6 in the development of CRS and ICANS after CD19.CAR-T cell therapy has been well established. Several strategies have been studied to decrease the risk of developing CRS and ICANS after CD19.CAR-T cell therapy, including prophylactic steroids and prophylactic tocilizumab. Oluwole et al. reported a series of 40 patients treated with prophylactic steroids starting on day 0 through day +2 after axi-cel CD19.CAR-T cell therapy and showed a modest improvement in grade of worst CRS and possible improvement in rate of ICANS.¹⁷ Initial clinical response rates were promising, but long term progression free survival and overall survival data is lacking. Caimi et al. reported a series of 20 patients treated with prophylactic tocilizumab prior to investigational CD19.CAR-T cell therapies and found a low rate of CRS, but persistent ICANS in this cohort. 15 Clinical response rates remained promising, though long term follow up was limited. The marginal impact on preventing ICANS with prophylactic tocilizumab has been postulated to be in part due to the mechanism of action of tocilizumab, acting on the IL-6 receptor rather than on circulating IL-6 directly.
Siltuximab is a chimeric murine antibody that binds directly to IL-6 and has been used effectively in the treatment of CRS, with guidelines recommending its use in CRS cases refractory to tocilizumab. 18,19 The inventors hypothesize that through direct binding of circulating IL-6, prophylactic siltuximab may lead improved prevention of ICANS as well as prevention of CRS as observed with tocilizumab. Here, the inventors propose a phase I study to evaluate the safety of administering a fixed dose of siltuximab prior to commercially available CD19.CAR-T cell therapies.

### 1.7 Rationale of Siltuximab Dosing

The phase I dose finding study of siltuximab in non-Hodgkin's lymphoma, multiple myeloma, and symptomatic Castleman disease found a recommended phase II dose of 12 mg/kg every 3 weeks based on sustained C-reactive protein (CRP) suppression and response rates in Castleman disease.²⁰ A subsequent phase II study evaluated siltuximab 11 mg/kg every 3 weeks compared to placebo in patients with Castleman disease, which led to Food and Drug Administration approval of siltuximab at this dose. 16 The half-life of siltuximab at this dose is approximately 21 days. Based on prolonged suppression of CRP and a half-life which would allow for coverage through the highest risk of developing CRS and ICANS, siltuximab will be dosed at 11 mg/kg prior to CD19.CAR-T therapy.

### 1.8 Rationale of Siltuximab for Treating CRS and ICANS

The therapy of choice for grade≥2 CRS is tocilizumab which binds to IL-6 receptor, but does not bind IL-6 itself. Multiple studies have shown that soluble IL-6 levels paradoxically rise in vivo following administration with tocilizimab while soluble IL-6 can be suppressed with siltuximab infusions.^{11,12} This has been hypothesized to be the mechanism in which CRS responds to tocilizumab, but ICANS does not respond to tocilizumab. The mainstay for treatment of ICANS grade≥2 has been high dose corticosteroids (often with a prolonged taper) which could lead to impaired cellular immunity as well as other side effects of prolonged steroid administration.
Grade 1 ICANS management is variable depending on the institution providing the cellular therapy and the cell therapy product utilized.
The inventors hypothesize that 1) treatment doses of siltuximab will prevent progression of symptoms of CRS in those who do develop CRS after CD19.CAR-T cell therapy and 2) treatment doses of siltuximab for low grade ICANS (grade 1 ICANS) may prevent progression of symptoms of ICANS in these patients without the need for corticosteroids. The potential mechanism for this is through direct IL-6 binding and clearance as opposed to IL-6 receptor binding as is the case with tocilizumab.
The use of siltuximab for treatment of CRS is currently recommended in the setting of tocilizumab refractory symptoms and siltuximab is currently under investigation for the treatment of CRS and ICANS in a separate clinical trial (NCT04975555). For these reasons, the inventors propose treatment dose of siltuximab for grade≥2 CRS, or grade 1 ICANS lasting >12 hours. Siltuximab treatment dose will also be investigated in combination with steroids in CRS with concurrent ICANS.

### 1.9 Correlative Studies

Several human studies have demonstrated the anti-lymphoma activity of anti-CD19.CAR-T cells.^{2,21} CD19.CAR-T cell expansion and persistence has been shown to correlate with clinical response. Inflammatory and cell growth-related cytokines and other biomarkers (i.e. interleukins, granzyme B, and ferritin) have shown variable correlation with the development of neurologic symptoms as well as cytokine release syndrome. A report by Kochenderfer and colleagues indicates interleukin-15 levels correlate with CAR-T cell product expansion and lymphoma response to this cellular therapy.²² In this study, correlative assays will include CD19.CAR-T cell expansion and persistence, cytokine changes, and immunomodulatory changes occurring after CD19.CAR-T cell therapy with siltuximab prophylaxis. The inventors will also investigate the role of apheresis, CD19.CAR-T, and pre-lymphodepletion immunophenotypes on CRS/ICANS development and CD19.CAR-T response.

### 2.0 Study Objectives

### 2.1 Primary Objectives

2.1.1 To determine the safety of siltuximab when given prior to CD19.CAR-T cells for prevention of cytokine release syndrome associated CAR-T cell therapy.

### 2.2 Secondary Objectives

2.2.1 To describe the safety profile of siltuximab as first line treatment of new onset grade≥2 CRS.
2.2.2 To estimate the incidence of all-grade CRS and grade≥3 CRS after CAR-T cell therapy with siltuximab prophylaxis.
2.2.3 To estimate the incidence of all grade ICANS and grade≥3 ICANS after CAR-T cell therapy with siltuximab prophylaxis.
2.2.4 To describe the response rates of grade≥2 CRS to treatment with a second dose of siltuximab.
2.2.5 To describe the disease response rates of lymphoma patients after CD19.CAR-T cell therapy with siltuximab prophylaxis.
2.2.6 To estimate the Progression Free Survival (PFS) in subjects treated with CD19.CAR-T cell therapy with siltuximab prophylaxis.
2.2.7 To estimate the Overall Survival (OS) in subjects treated with CD19.CAR-T cell therapy with siltuximab prophylaxis.

### 2.3 Exploratory Objectives

2.3.1 To describe CD19.CAR-T cell expansion and persistence following treatment with siltuximab prophylaxis.
2.3.2 To describe changes in plasma cytokine concentrations in patients treated with CD19.CAR-T cells with prophylactic siltuximab.
2.3.3 To describe changes in peripheral blood immunophenotypes in patients treated with CD19.CAR-T cells with prophylactic siltuximab.
2.3.4 To examine the role of complement activation on development and persistence of ICANS.
2.3.5 To examine the correlation between apheresis product, infusional CAR-T product and pre-lymphodepletion immunophenotypes with CRS/ICANS and CD19.CAR-T clinical response.

### 3.0 Study Endpoints

### 3.1 Primary Endpoint

3.1.1 Safety will describe frequency and nature of adverse events associated with siltuximab prophylaxis prior to CD19.CAR-T cell therapy using CTCAE v5.0.

### 3.2 Secondary Endpoints

3.2.1 Safety will describe frequency and nature of adverse events associated with siltuximab treatment of CRS and/or ICANS using CTCAE v5.0.
3.2.2 Incidence of all grade CRS and grade≥3 CRS following CD19.CAR-T cell therapy with siltuximab prophylaxis according to ASTCT consensus grading criteria.
3.2.3 Incidence of all grade ICANS and grade≥3 ICANS following CD19.CAR-T cell therapy with siltuximab prophylaxis according to ASTCT consensus grading criteria.
3.2.4 Percentage of participants who achieve resolution of CRS defined as absence of symptoms leading to diagnosis of CRS at 24 hours from treatment with Siltuximab
3.2.5 Objective response rate (CR+PR) at day 30 following CD19.CAR-T cell therapy.
3.2.6 PFS, defined as the duration of time from CD19.CAR-T infusion to clinical progression or death as a result of any cause using Lugano criteria.
3.2.7 OS, defined as the duration of time from CD19.CAR-T infusion to date of death as a result of any cause.

### 3.3 Exploratory Endpoints

3.3.1 CD19.CAR-T cell expansion and persistence from Day 0 to Day 30 measured by flow cytometry in the peripheral blood.
3.3.2 Plasma cytokine levels from Day 0 to Day 30 in the peripheral blood measured by enzyme linked immunoassays.
3.3.3 Peripheral blood immunophenotype changes from Day 0 to Day 30 in peripheral blood measured by spectral flow cytometry.
3.3.4 Peripheral blood LDH, haptoglobin, schistocytes, and C5b-9 levels at Day 0, time of diagnosis of ICANS, every 2 days until ICANS resolution, and at resolution of ICANS.
3.3.5 Immunophenotype from any residual apheresis product, CD19.CAR-T product, and pre-lymphodepletion peripheral blood by spectral flow cytometry.

### 4.0 Subject Eligibility

In order to participate in this study a subject must meet al.L of the eligibility criteria outlined below.

### 4.1 Inclusion Criteria for Prophylaxis

4.1.1 Written informed consent obtained to participate in the study and HIPAA authorization for release of personal health information.
4.1.2 Age≥18 years at the time of consent.
4.1.3 Diagnosis of non-Hodgkin lymphoma.
4.1.4 Eligible for standard of care CD19.CAR-T cell therapy including axicabtagene ciloleucel, tisagenlecleucel, brexucabtagene autoleucel and lisocabtagene maraleucel).
4.1.5 Subjects with hepatitis B virus (HBV) can be included if on suppressive antiviral therapy and have no detectable viral load.
4.1.6 Subjects with hepatitis C virus (HCV) can be included if HCV RNA viral load is undetected.
4.1.7 Measurable disease of >1.5 cm in diameter and/or bone marrow involvement.
4.1.8 Demonstrate adequate organ function.
4.1.9 Table 1 below; all screening labs to be obtained within 14 days prior to initiating study treatment.

TABLE 1

| | |
|---|---|
| System | Laboratory Value |

Hematological*

| | |
|---|---|
| White blood cell count | ≥1.0 × 10⁹/L |
| Hemoglobin | ≥8.0 × 10⁹/L |
| Platelets | ≥50 × 10⁹/L |

Renal*

| | |
|---|---|
| Creatinine | ≤2.0 × ULN |

Hepatic*

| | |
|---|---|
| Bilirubin | ≤2.0 × upper limit of normal (ULN). |
| | Subjects with Gilbert's syndrome may |
| | be enrolled despite a total bilirubin |
| | level >2.0 mg/dL if their conjugated |
| | bilirubin is <2.0 × ULN) |
| Aspartate | ≤3.0 × ULN |
| aminotransferase | |
| (AST) | |
| Alanine | ≤3.0 × ULN |
| aminotransferase | |
| (ALT) | |

*Note:
Hematology and other lab parameters that are ≤ grade 2 BUT still meet criteria for study entry are allowed.
Furthermore, changes in laboratory parameters during the study should not be considered adverse events unless they meet criteria for dose modification(s) of study medication outlined by the protocol and/or worsen from baseline during therapy.

4.1.10 Females of childbearing potential must have a negative serum pregnancy test within 3 days prior to registration. NOTE: Females are considered of childbearing potential unless they are surgically sterile (have undergone a hysterectomy, bilateral tubal ligation, or bilateral oophorectomy) or they are naturally postmenopausal for at least 12 consecutive months. Documentation of postmenopausal status must be provided.
4.1.11 Females of childbearing potential must be willing to abstain from heterosexual activity or to use 2 forms of effective methods of contraception from the time of informed consent until 12 months after treatment the last dose of siltuximab. The two contraception methods can be comprised of two barrier methods, or a barrier method plus a hormonal method or an intrauterine device that meets <1% failure rate for protection from pregnancy in the product label.
4.1.12 Male subjects with female partners must have had a prior vasectomy or agree to use an adequate method of contraception (i.e., double barrier method: condom plus spermicidal agent) starting with the first dose of study therapy through 12 months after the last dose of siltuximab.
4.1.13 Subjects with prior or concurrent malignancy whose natural history or treatment does not have the potential to interfere with the safety or efficacy assessment of the experimental regimen are eligible for the trial.
4.1.14 Subject is willing and able to comply with study procedures based on the judgement of the investigator or protocol designee.

### 4.2 Exclusion Criteria for Prophylaxis

4.2.1 Subjects requiring daily corticosteroids at a dose>10 mg of prednisone per day (or equivalent). Pulsed steroids for disease control are acceptable.
4.2.2 Subjects who are planned to receive prophylactic steroids for mitigating CRS/ICANS before, during, or after CD19.CAR-T infusion.
4.2.3 Active central nervous system or meningeal involvement by lymphoma. Subjects with symptoms of possible CNS disease should undergo CNS workup with MRI brain and diagnostic lumbar puncture prior to enrollment on this study. Subjects with a history of CNS or meningeal involvement must be in a documented remission by CSF evaluation and contrast-enhanced MRI imaging for at least 90 days prior to registration.
4.2.4 Patients with history of clinically relevant and active CNS pathology such as epilepsy, seizure disorders, paresis, aphasia, uncontrolled cerebrovascular disease, severe brain injuries, dementia and Parkinson's disease
4.2.5 HIV seropositivity
4.2.6 Pregnant or breastfeeding (NOTE: breast milk cannot be stored for future use while the mother is being treated on study and lactating females must agree to not breastfeed while taking study drugs).
4.2.7 Uncontrolled concomitant illness including, but not limited to, symptomatic congestive heart failure (New York Heart Association (NYHA) Class III or IV), unstable angina pectoris, myocardial infarction within 1 month prior to enrollment, uncontrolled cardiac arrhythmias, uncontrolled seizures, or severe non compensated hypertension (Systolic blood pressure≥180 mmHg or Diastolic blood pressure≥120 mmHg).

### 4.3 Inclusion Criteria for Siltuximab Treatment of CRS/ICANS

4.3.1 Any subject with grade 1 ICANS lasting >12 hours.
4.3.2 Any subject with grade≥2 CRS after CD19.CAR-T cell therapy.
4.3.3 Any subject with grade≥2 CRS after CD19.CAR-T cell therapy with concurrent ICANS.

### 4.4 Exclusion Criteria for Siltuximab Treatment of CRS/ICANS

4.4.1 Subjects with CRS and no evidence of ICANS who require dexamethasone (or any steroid) due to severity of CRS as determined by the investigators.
4.4.2 Prior history of a dose limiting toxicity (see section 6.5) due to siltuximab infusion according to NCI-CTCAE criteria v5.0.

### 4.5 Inclusion Criteria for Second Siltuximab Treatment Dose for CRS/ICANS

4.5.1 Subjects meeting criteria for a treatment dose in Section 4.3 who have received a treatment dose and have no change in the CRS or ICANS grade after 12 hours.

### 4.6 Exclusion Criteria for Second Siltuximab Treatment Dose for CRS/ICANS

4.6.1 Subjects with CRS and no evidence of ICANS who require dexamethasone (or any steroid) due to severity of CRS as determined by the investigators.
4.6.2 Prior history of a dose limiting toxicity (see section 6.5) due to siltuximab infusion according to NCI-CTCAE criteria v5.0.

### 5.0 Treatment Plan

### 5.1 Treatment Dosage and Administration

TABLE 2
Treatment Regimen

| | Premedications; | | | Infusion | |
|---|---|---|---|---|---|
| Agent | Precautions | Dose | Route | Duration | Schedule |
| Siltuximab | Per Institutional | 11 mg/kg reconstituted | IV | 60 min | Prophylaxis |
| | Standards | in 250 ml Dextrose 5% | | | Day 0, 1 hour prior to |
| | | water solution. | | | CD19.CAR-T infusion |
| | | | | | Treatment |
| | | | | | Administer 11 mg/kg |
| | | | | | ** second treatment dose |
| | | | | | available at 12 hours |
| | | | | | after the first treatment |
| | | | | | dose if CRS and/or ICANS |
| | | | | | remains the same grade |
| | | | | | ** decisions on treatment |
| | | | | | dose siltuximab will be |
| | | | | | made by the inpatient |
| | | | | | attempting responsible |
| | | | | | for the subject's care. |
| | | | | | It is recommended that the |
| | | | | | study team be consulted |
| | | | | | about treatment dose |
| | | | | | administration. |

### 5.1.1 Managing Infusion Related Reactions and Hypersensitivity

During IV infusion of siltuximab, mild-to-moderate infusion reactions may improve following slowing of or stopping the infusion. Upon resolution of the reaction, reinitiating the infusion at a lower infusion rate and therapeutic administration of antihistamines, acetaminophen, and corticosteroids may be considered. For patients who do not tolerate the infusion following these interventions, siltuximab should be discontinued. During or following infusion, treatment with siltuximab should be discontinued in patients who have severe infusion-related hypersensitivity reactions (e.g., anaphylaxis).

### 5.1.2 Dose Modifications/Delays for Siltuximab

Because this trial involves single interventions (i.e. one dose of siltuximab prophylaxis and potentially an additional 1-2 doses of siltuximab treatment), there is no plan for dose modification of the prophylactic dose or the treatment dose. Any patient with a DLT toxicity according to Section 6.5 will not be eligible for subsequent treatment doses.

### 5.2 Concomitant Medications/Treatments/Supportive Care Allowed

The following concomitant medications/treatment and supportive cares are allowed:
   Infusion premedication for Siltuximab: antipyretics, histamine H1 or H2 blockers, glucocorticoids.
   Lymphodepleting chemotherapy, including but not limited to cyclophosphamide, fludarabine, and/or bendamustine.
   CAR-T cell therapy.
   Antibiotics (antifungal, antiviral, antimicrobial).
   Antiemetic
   Other supportive medications at the discretion of provider.
   Corticosteroid administration for the treatment of CRS with concurrent ICANS is allowed. If a subject has CRS and no evidence of ICANS and receives steroid treatment, they will be ineligible for a Siltuximab treatment dose. Steroids for worsening grade CRS and/or ICANS are allowed after treatment dose Siltuximab.
   Siltuximab may restore inflammation-mediated CYP450 inhibition, and therefore co-administration with agents that are substrates of CYP3A4 should be done with caution and with the understanding that siltuximab could decrease the concentration of these drugs
   Live, attenuated vaccines should not be given within 4 weeks of initiating Siltuximab infusion.

### 5.3 Prohibited Medications/Treatments

There are no prohibited medications in this trial. Please see section 6.3 regarding corticosteroid administration and other precautions.

### 5.4 Definition of Dose Limiting Toxicity

Management of common toxicities are outlined in section 7.0.
In this study, a dose limiting toxicity (DLT) includes those adverse events graded according to NCI Common Terminology Criteria for Adverse Events (CTCAE) version 5.0, with the exception of cytokine release syndrome and ICANS to be graded using criteria provided in Appendices C, D and E.
To be considered DLTs, adverse events must be possibly secondary to siltuximab infusion, must not be considered definitely associated with CAR-T cell therapy or lymphodepletive therapy and occur during the first 30 days after infusion and meet the following criteria:
   1. Grade 4 life-threatening toxicity.
   2. Grade 3 non hematologic toxicity involving a vital organ system, with the following exceptions:
      Laboratory abnormalities without associated symptomatology or clinical consequence that resolve in less than 7 days;
      ·Laboratory abnormalities associated with tumor lysis syndrome.

### 5.5 Definition of Siltuximab Treatment Failure

Siltuximab failure will be defined for any subject who received a treatment dose of Siltuximab for CRS/ICANS and develops worsening grade of either CRS or ICANS.
If a subject maintains the same CRS or ICANS grade, this will not be defined as treatment failure. Additionally, if the subject goes on to receive any additional therapy such as steroids, tocilizumab, or other therapy and the CRS and/or ICANS grade remains the same, this will not be defined as treatment failure.

### 5.6 Duration of Study Therapy

In the absence of treatment delays due to adverse events, follow up will continue until:
   DLT window will end on Day 30 assuming no ongoing toxicity associated with Siltuximab. (Subjects treated for CRS/ICANS will be followed until 30 days after their last infusion).
   Disease progression at any point during therapy
   Subject is lost to follow up
   1 year from CD19.CAR-T infusion.

### 5.7 Duration of Follow Up

Subjects will be followed for acute toxicity and efficacy for 30 days after prophylactic treatment has been discontinued or until death, whichever occurs first. In patients that receive siltuximab for treatment dose of CRS and/or ICANS, follow up will continue until 30 days have elapsed since the last infusion of siltuximab.
The clinical course of each event associated with siltuximab will be followed until resolution, stabilization, or until it has been determined that the study treatment or participation is not the cause.
Serious adverse events that are considered related to siltuximab and still ongoing at the end of the study period will necessitate follow-up to determine the final outcome. Any serious adverse event that occurs after the study period and is considered to be possibly related to the study treatment or study participation will be recorded and reported immediately.
All subjects will be followed for progression and survival per standard of care and institutional follow up policies after the 30 day mark for up to 1 year.

### 6.0 Drug Information

### 6.1 Siltuximab

### 6.1.1 How Supplied

Siltuximab is supplied in single-use vials containing 100 mg or 400 mg of siltuximab as a sterile, lyophilized formulation for reconstitution and IV infusion. Each single-use vial contains siltuximab powder (100 mg or 400 mg) for concentrate for solution for infusion. After reconstitution the solution contains 20 mg siltuximab per mL.

### 6.1.2 Preparation

Calculate the dose (mg), total volume (mL) of reconstituted siltuximab solution required and the number of vials needed. The recommended needle for preparation is 21-gauge 1½ inch (38 mm). Infusion bags (250 mL) must contain dextrose 5% in water and must be made of made of polyvinyl chloride (PVC), or polyolefin (PO), or polypropylene (PP), or polyethylene (PE). Alternatively, PE bottles may be used.
Allow the vial(s) of siltuximab to come to room temperature over approximately 30 minutes. Siltuximab should remain at room temperature for the duration of the preparation.
Gently swirl (DO NOT SHAKE or VORTEX or SWIRL VIGOROUSLY) the reconstituted vials to aid the dissolution of the lyophilized powder. Do not remove contents until all of the solids have been completely dissolved. The lyophilized powder should dissolve in <60 minutes.
Once reconstituted, and prior to further dilution, inspect the vials for particulate matter and discoloration prior to dose preparation. Do not use if visibly opaque or foreign particles and/or solution discoloration are present. The reconstituted product should be kept for no >2 hours prior to addition into the IV infusion bag.
Dilute the total volume of the reconstituted siltuximab solution dose to 250 ml with sterile dextrose 5%, by withdrawing a volume equal to the volume of reconstituted siltuximab from the dextrose 5%, 250 ml bag. Slowly add the total volume (mL) of reconstituted siltuximab solution to the 250 mL infusion bag. Gently invert the bag to mix the solution.

TABLE 3
Siltuximab reconstitution instructions

| | Amount of | |
|---|---|---|
| | Sterile Water | |
| | for Injection, | Post- |
| | USP required | reconstitution |
| Strength | for reconstitution | concentration |
| 100 mg | 5.2 mL | 20 mg/mL |
| vial | | |
| 400 mg | 20 mL | 20 mg/mL |
| vial | | |

### 6.1.3 Storage and Handling

Siltuximab should be stored in a refrigerator at 2° C. to 8° C. (36° F. to 46° F.). Do not freeze. Store in the original package in order to protect from light. The reconstituted product should be kept for no >2 hours prior to addition into the IV infusion bag.

### 6.1.4 Route of Administration

### Intraveneous.

The infusion should be completed within 6 hours of the addition of the reconstituted solution to the infusion bag. Administer the diluted solution over a period of 1 hour using administration sets lined with PVC or polyurethane (PU), or PE containing a 0.2-micron inline polyethersulfone (PES) filter. Siltuximab does not contain preservatives; therefore, do not store any unused portion of the infusion solution for reuse.

### 6.1.5 Method of Administration

Siltuximab will be administered via IV infusion over a 1-hour period (+/−30 minutes). If the subject experience an infusion reaction, the infusion will be stopped, the subject will be treated with supportive care measures (i.e. corticosteroids, antihistamines, etc), see Section 6.1.1. Subjects with resolution of symptoms attributable to infusion reactions can resume infusion at a slower rate.
   6.1.6 Adverse Events Associated with Siltuximab
The most frequent ADRs (in >20% of patients) during treatment with siltuximab in the MCD clinical trials were upper respiratory tract infection, pruritus, rash, arthralgia, and diarrhea. The most serious ADR associated with the use of siltuximab was anaphylactic reaction (2%).
For the purpose of safety reporting in clinical trials with siltuximab, irrespective of any other combination therapy or disease being treated with study drug, the serious adverse reactions provided in Table 7 (Section 7.10) of the Siltuximab IB (v.13) should be considered as expected for siltuximab.

### 6.1.7 Anaphylaxis and Infusion Related Reactions

Approximately 945 patients have been treated with siltuximab. Of these, one patient experienced an anaphylactic reaction. Data from 254 patients treated with siltuximab monotherapy forms the basis of the safety evaluation of infusion related reactions. Infusion related reactions were reported in 5.1% of these patients. Symptoms of infusion reactions consisted of back pain, chest pain or discomfort, nausea and vomiting, flushing, erythema, and palpitations.

### 6.1.8 Immunogenicity

In clinical studies, including single-agent and combination studies, 4 of 432 (0.9%) evaluable patients tested positive for anti-siltuximab antibodies. Further immunogenicity analyses were conducted for all positive samples from the 4 patients with detectable anti-siltuximab antibodies. None of these patients had neutralizing antibodies. No evidence of altered safety or efficacy was identified in the patients who developed antibodies to siltuximab.

### 7.0 Clinical Assessments

Clinical assessments will be performed as outlined in the Time and Events Table.

### 7.1.1 Informed Consent

The Investigator must obtain documented consent from each potential subject prior to participating in a clinical trial.

### 7.1.2 General Informed Consent

Consent must be documented by the subject's dated signature or by the subject's legally acceptable representative's dated signature on a consent form along with the dated signature of the person conducting the consent discussion.
A copy of the signed and dated consent form should be given to the subject before participation in the trial.
The initial informed consent form, any subsequent revised written informed consent form and any written information provided to the subject must receive the IRB/ERC's approval/favorable opinion in advance of use. The subject or his/her legally acceptable representative should be informed in a timely manner if new information becomes available that may be relevant to the subject's willingness to continue participation in the trial. The communication of this information will be provided and documented via a revised consent form or addendum to the original consent form that captures the subject's dated signature or by the subject's legally acceptable representative's dated signature.
Specifics about a trial and the trial population will be added to the consent form template at the protocol level.
The informed consent will adhere to IRB/ERC requirements, applicable laws and regulations and Sponsor requirements.

### 7.1.3 Inclusion/Exclusion Criteria

All inclusion and exclusion criteria will be reviewed by the investigator or qualified designee to ensure that the subject qualifies for the trial.

### 7.1.4 Concomitant Medications

All concomitant medication and concurrent therapies will be documented. Dose, route, unit frequency of administration, and indication for administration and dates of medication will be captured. Follow-up medication review should include any anti-cancer treatments that have started after the end of study treatment.

### 7.1.5 Demographics

Demographic information including date of birth, gender, race will be recorded.

### 7.1.6 Medical History

Relevant medical history, including history of current disease, other pertinent history, and information regarding underlying diseases will be recorded at Screening and a focused medical history on symptoms/toxicity will be performed thereafter.

### 7.1.7 Physical Examination

A complete physical examination including height (at screening only), weight, Performance status ECOG or Karnofsky and vital signs (i.e., temperature and blood pressure) will be performed at Screening.
New abnormal physical exam findings must be documented and will be followed by a physician or other qualified staff at the next scheduled visit.

### 7.1.8 CRS Assessment

Clinician will perform CRS assessment based on current symptoms and laboratory findings per Appendix C.

### 7.1.9 Neurotoxicity Assessment

Clinician will perform ICE neurotoxicity assessments per Appendix D and Lee et al. 2019, supra.

### 7.1.10 Toxicity Assessment (Adverse Events)

Events should be assessed per NCI-CTCAE criteria v5.0. Information regarding occurrence of adverse events will be captured throughout the study. Duration (start and stop dates), severity/grade, outcome, treatment and relation to study drug will be recorded in the electronic case report form (eCRF).

### 7.1.11 Disease Assessment

Baseline disease assessment should be obtained within 28 days of the screening visit, and then conducted at 30 days after CD19.CAR-T infusion (+/−3 days).

### 7.2 Clinical Laboratory Assessments

### 7.2.1 Hematology

Blood will be obtained and sent to the clinical site hematology lab for a complete blood count (hemoglobin, hematocrit, red blood cell count, white blood cell count, white blood cell differential, and platelet count).

### 7.2.2 Serum Chemistry and Liver Function Tests

Blood will be obtained and sent to the clinical site chemistry lab for determination of serum sodium, potassium, chloride, bicarbonate, glucose, BUN, creatinine, aspartate aminotransferase (AST/SGOT), alanine aminotransferase (ALT/SGPT), alkaline phosphatase, total bilirubin, indirect bilirubin, albumin, LDH, C-reactive protein, and ferritin.

### 7.2.3 Virus Testing

### 7.2.3.1 Hepatitis B Virus (HBV) and Hepatitis C Virus (HCV)

HBV and HCV. HBV core Ab, HBV surface Ab, HBV surface Ag. Subjects with positive HBV surface Ag will require HBV PCR to rule out active HBV infection. Subjects with HBV PCR positivity will not be eligible until PCR is undetectable. Subjects with a positive HBV core Ab may continue on the trial using prophylactic entecavir (recommend continuing for 6 months after completion of therapy). If subjects are found to have a positive HCV Ab, active HCV will be evaluated with HCV PCR. Subjects negative for HCV PCR may participate in the study with monitoring as long as liver function tests.

### 7.2.3.2 Human Immunodeficiency Virus (HIV)

HIV screening with Ab/Ag testing. Patients with positive HIV Ab/Ag are not eligible.

### 7.2.4 Pregnancy Test

A urine or serum pregnancy test will be obtained from female subjects who are of childbearing potential prior to their participation in the study.

### 7.3 Correlative Studies

The following investigations will be used further characterize the tumor microenvironment, peripheral blood immunophenotype changes, and predictive factors for developing ICANS in subjects being treated with siltuximab at the time-points indicated in the Time and Events Table.

### 7.3.1 CD19.CAR-T Expansion

Peripheral blood will be evaluated for CD19.CAR-T expansion and persistence at the following time points: Days 1, 2, 3, 4, 5, 6, 7, 14, 21, and 30. CD19.CAR-T detection will be performed by flow cytometry.

### 7.3.2 Immunomodulation

Peripheral blood will be evaluated for immune cell subset changes at the following time points: pre-lymphodepletion, Day 0, 3, 7, 14, 21, and 30. Additionally, any available samples from the apheresis product and discarded CD19.CAR-T product will be collected and analyzed. Immunomodulation will be assessed by spectral flow cytometry.

### 7.3.3 Cytokine Levels

Plasma from the peripheral blood will be evaluated for cytokine level changes at the following time points: pre-lymphodepletion, Day 0, 3, 7, 14, 21, 30 at initiation of CRS/ICANS, through treatment of CRS/ICANS and at resolution of CRS/ICANS. Cytokine levels will be assessed by the Human Cytokine/Chemokine 48-Plex Discovery Assay (Eve Technologies).

### 7.3.4 Complement Activation

Plasma from the peripheral blood will be evaluated for evidence of hemolysis (LDH, haptoglobin, schistocytes) and complement activation (C5b-9 levels) at the following time points: pre-lymphodepletion, Day 0, 7, 14, 21, at initiation of ICANS and at resolution of ICANS.

### 8.0 Evaluations and Assessments

TABLE 4
8.1 Time and Events Table

| | | Pre- | Day | Days | Day | Day | Day | Long-term |
|---|---|---|---|---|---|---|---|---|
| Assessments | Screening¹ | Lymphodepletion¹ | 0¹ | 1-7¹ | 14¹ | 21¹ | 30¹ | Follow-up |
| Informed consent | X | | | | | | | |
| History⁴ | X | X | X | X | X | X | X | |
| Physical exam⁴ | X | X | X | X | X | X | X | |
| Eligibility verification | X | | | | | | | |
| ECOG/KPS performance | X | X | | | | | | |
| status | | | | | | | | |
| Radiographic tumor | X | | | | | | X² | X³ |
| evaluation⁵ | | | | | | | | |
| Pregnancy test⁶ | X | | X | | | | | |
| Hematology⁷ | X | X | X | X | X | X | X | |
| Serum chemistries⁸ | X | X | X | X | X | X | X | |
| Toxicity assessment⁹ | X | X | X | X | X | X | X | |
| CRS grading¹⁰ | | | | X | X | X | X | |
| ICANS grading¹¹ | | | | X | X | X | X | |
| Virus Testing¹² | X | | | | | | | |
| HIV Testing¹³ | X | | | | | | | |
| Concomitant med review | X | X | X | X | X | X | X | X¹⁷ |
| Siltuximab (11 mg/kg) ¹⁴ | | | X | | | | | |
| CD19.CAR-T cell infusion | | | X | | | | | |
| Archival tissue¹⁵ | X | | | | | | | |
| Blood sample, correlative¹⁶ | | X | X | X | X | X | X | |
| Blood sample, CRS/ICANS | | | | X | | | | |
| correlative¹⁶ | | | | | | | | |
| Survival | | | | | | | X | X |

Abbreviations:
C = cycle,
D = day,
EOT = end of treatment
Footnotes to the Time and Events Table
¹Unless otherwise specified, study visits have a window of +/−3 days. Unless otherwise noted screening assessments should be completed within 30 days prior to the start of study treatment. Screening labs should be completed within 30 days of start of study treatment.
²The window for Day 30 imaging will be +/−3 days.
³All long-term follow-up scans and visits will be per standard of care and intuitional protocols. Radiographic images performed as standard of care will be collected for determination of progression.
⁴A complete physical exam and medical history at screening with abbreviated physical exams and focused medical history thereafter.
⁵Tumor imaging should include PET/CT of the skull to thigh. If a PET scan is not approved by insurance, CT Chest, Abdomen, and Pelvis is an acceptable alternative imaging technique. Imaging for screening should be completed within 28 days of the start of study treatment.
⁶Serum or urine pregnancy test required within 72 hours of start of study therapy.
⁷Hematology: CBC with differential
⁸Serum chemistry: potassium, sodium, calcium, creatinine, magnesium, phosphorus, BUN, albumin, ALT, AST, Alk Phos, total bilirubin, indirect bilirubin, total protein, LDH, ferritin, and CRP. These will be collected at each timepoint listed.
⁹Toxicity assessment by CTCAE v5.0. May be performed in person or remotely.
¹⁰CRS grading will be performed per ASTCT consensus grading (see Appendix C)
¹¹ICANS grading will be performed per ASTCT consensus grading (see Appendix D)
¹²HBV and HCV. HBV core Ab, HBV surface Ab, HBV surface Ag. Subjects with positive HBV surface Ag will require HBV PCR to rule out active HBV infection. Subjects with HBV PCR positivity will not be eligible until PCR is undetectable. Subjects with a positive HBV core Ab may continue on the trial using prophylactic entecavir (recommend continuing for 6 months after completion of therapy). If subjects are found to have a positive HCV Ab, active HCV will be evaluated with HCV PCR. Subjects negative for HCV PCR may participate in the study with monitoring as long as liver function tests.
¹³HIV screening with Ab/Ag testing.
¹⁴ Siltuximab prophylaxis infusion (11 mg/kg) will be administered intravenously over 1 hour, starting 1 hour prior to CD19.CAR-T infusion. Subjects eligible for a treatment dose of siltuximab due to developing grade ≥2 CRS or grade 1 ICANS lasting 12 hours as outlined in the inclusion/exclusion criteria will be eligible for a treatment infusion of siltuximab 11 mg/kg to be delivered intravenously over 1 hour. If CRS or ICANS grade remains the same at 12 hours, a second treatment infusion of siltuximab 11 mg/kg may be administered intravenously over 1 hour.
¹⁵Fixed paraffin-embedded blocks and/or slides from the diagnostic specimen prior to CD19.CAR-T cell therapy will be requested for research purposes. Additionally, any residual apheresis product (from collection prior to CAR-T manufacturing) or unused CD19.CAR-T cell product that is available will be collected for research purposes.
¹⁶Blood samples collected for correlative studies. Collection window is +/−3 days. If a subject develops CRS and/or ICANS requiring siltuximab infusion, the inventors will also plan to collect blood samples prior to siltuximab treatment infusion, at 12 hours, 24 hours, 48 hours, and resolution of CRS/ICANS (all treatment collections will have a window period of +/−6 hours). See section 8.3 for correlative studies.
¹⁷Concomitant medications in follow up includes subsequent lines of therapy.

### 8.2 Assessment of Safety

Any subject who receives at least one dose of study therapy on this protocol will be evaluable for toxicity. Each subject will be assessed periodically for the development of any toxicity according to the Time and Events Table. Toxicity will be assessed according to the NCI CTCAEv5.0.

### 8.3 Assessment of Efficacy

All subjects who receive Siltuximab prophylaxis prior to CD19.CAR-T cell therapy will be evaluable for efficacy analysis. To assess disease response, a PET/CT or CT Chest, Abdomen, and Pelvis will be performed at 30 days (+/−3 days). Subsequent disease assessments will be performed per standard of care and institutional standards. Please consult Appendix A. Revised response criteria for lymphoma23 for details on disease assessment using Lugano criteria.

### 9.0 Adverse Events

### 9.1 Definitions

### 9.1.1 Unanticipated Problems Involving Risks to Subjects or Others (UPIRSO)

An incident, experience, or other problem that meets all of the following criteria:
   unexpected (in terms of nature, severity, or frequency) given (a) the research procedures that are described in the protocol-related documents, such as the IRB-approved research protocol and informed consent document; and (b) the characteristics of the subject population being studied;
   related or possibly related to participation in the research (in this guidance document, possibly related means there is a reasonable possibility that the incident, experience, or outcome may have been caused by the procedures involved in the research); and
   suggests that the research places subjects or others at a greater risk of harm (including physical, psychological, economic, or social harm) than was previously known or recognized, even if no harm actually occurred. Unanticipated problems involving risks to subjects or others may be medical or non-medical in nature and include—but are not limited to—serious, unexpected, and related adverse drug events and unanticipated adverse device effects (see below).

### 9.1.2 Adverse Event (AE)

Any undesirable and unintended (although not necessarily unexpected) effect occurring as a result of interventions, interactions, or collection of identifiable private information in research. In medical research, any untoward physical or psychological occurrence in research, including abnormal laboratory finding, symptom, or disease temporally associated with the use of (although not necessarily related to) a medical treatment or procedure. Adverse events involving drugs are also referred to as adverse drug experiences.

### 9.1.3 Serious Adverse Event (SAE)

An adverse event that is fatal or life threatening, permanently disabling, requires or prolongs hospitalization, or results in significant disability, congenital anomaly, or birth defect. Important medical events that may not result in death, be life-threatening, or hospitalization may be considered serious when, based upon appropriate medical judgment, they may jeopardize the patient and may require medical or surgical intervention to prevent one of the outcomes above.

### 9.1.4 Unexpected AE

An adverse event that has not been previously observed or is not consistent in nature, severity, or frequency with existing risk information, such as in the investigator's brochure, research protocol, consent form, or other available information (e.g., IND application for an investigational drug).

### 9.1.5 Related

Associated or having a timely relationship with; a reasonable possibility exists that an outcome may have been caused or influenced by the event in question (e.g., administration of a study drug), although an alternative cause/influence may also be present. AEs will be reported as related or not related.

### 9.1.6 Unrelated

Unassociated or without a timely relationship; evidence exists that an outcome is definitely related to a cause other than the event in question.

### 10.0 Statistical Considerations

### 10.1 Study Design/Study Endpoints

This prospective single center phase I trial is designed to evaluate the safety of siltuximab prophylaxis prior to standard of care CD19.CAR-T cell therapy. The DLT observation period will be defined as Day 0 through Day 30. There is no plan for escalating or de-escalating the dose of siltuximab.
The sample size of 10 subjects has been selected based on feasibility of enrolling patients in a 6-month period of time.
The primary endpoint will be the frequency and nature of adverse events associated with siltuximab prophylaxis prior to CD19.CAR-T cell therapy using CTCAE v5.0. A DLT will be defined as any grade 4 life-threatening toxicity or grade 3 non hematologic toxicity involving a vital organ system (must be suspected to be possibly related to siltuximab infusion, not be considered definitely associated with CAR-T cell therapy or lymphodepletive therapy). The following will not be considered a DLT:
1) Laboratory abnormalities without associated symptomatology or clinical consequence that resolve in less than 7 days.
2) Laboratory abnormalities associated with tumor lysis syndrome.
3) CRS and ICANS.
The primary analysis of safety and clinical response will be performed after all subjects have cleared Day 30 of treatment with initial siltuximab dose. Subjects will be followed for up to 1 year for disease response.

### 10.2 Stopping Rules for Safety

Sequential boundaries will be used to monitor excessive toxicity using rules presented in Table 5 below. The accrual will be halted if the numbers of DLTs as defined in section 6.5 are observed in b, or more out of n patients within 30 days of DLT observation period. Additionally, accrual will be halted for any grade 5 adverse event assessed as possibly related to siltuximab and any adverse events related to siltuximab that delay CAR-T administration by more than 12 hours or prevent CAR-T administration. This Pocock-type stopping boundary yields at most 5% probability of crossing the boundary when the true rate of DLT is equal to the acceptable rate of 20%.

TABLE 5
Stopping Rules for Safety

| | Number of Subjects, n | | | | | | | | | |
|---|---|---|---|---|---|---|---|---|---|---|
| | 1 | 2 | 3 | 4 | 5 | 6 | 7 | 8 | 9 | 10 |
| Boundary, bₙ | — | — | 3 | 3 | 4 | 4 | 5 | 5 | 5 | 6 |

### 10.3 Sample Size, Accrual and Duration of Accrual

10 evaluable patients based on feasibility. Accrual duration is anticipated to require 6-12 months.

### 10.3.1 Accrual Duration

With an anticipated accrual rate of 1-2 patients per month, the study is expected to finish enrollment in 6-12 months.

### 10.4 Data Analysis Plans

### 10.4.1 Safety, ORR, PFS, and OS

Adverse events (AEs) will be described and classified per the NCI CTCAE v5.0 guidelines. The maximum grade of each type of toxicity will be extracted for each patient, and frequency counts will be tabulated to determine toxicity patterns, especially for grade 3 or above adverse events.
Since the primary endpoint is the safety of siltuximab as prophylaxis, the AE data associated with prophylaxis will be summarize for every patient: for patients receiving siltuximab only as prophylaxis, all AEs occur during the 30-day DLT observation period will be considered; for patients receiving siltuximab both as prophylaxis and as treatment, only the AEs occur before the treatment start will be considered for the primary endpoint.
The secondary endpoint is the safety of siltuximab as treatment for CRS and/or ICANS for patients requiring siltuximab treatment, for this subgroup of patients, the AEs post treatment start will be summarized for the secondary endpoint.
Secondary endpoints will also include incidence of all grade and grade≥3 CRS and/or ICANS following CD19.CAR-T cell therapy with siltuximab prophylaxis, percentage of subject who achieve resolution of CRS at 24 hours from treatment with siltuximab, and estimates of ORR, PFS, and OS.
The ORR (CR+PR) will be calculated at the initial 30 day imaging assessment and 95% confidence interval computed.
Progression-free survival will be calculated from the date of treatment initiation to date of progression or death, whichever occurs first, censoring patients who are alive without progression at time of last known follow-up. Overall survival will be calculated from date of treatment initiation to date of death due to all causes, and censoring alive patients at date of last known follow-up. PFS and OS will be estimated using the Kaplan-Meier method.
All demographic and analytic data will be summarized by descriptive statistics. Categorical data will be summarized using frequency tables, while summary statistics such as means, medians, standard deviation, range, etc. will be provided for continuous data.

### 10.4.2CD19.CAR-T Expansion, Cytokines, Immunophenotypes, Complement Activation

CD19.CAR-T cell expansion and persistence, plasma cytokine levels, peripheral blood immunophenotype changes, and complement activation assays will be exploratory in nature. The inventors will calculate a mean and/or median value for each measurement time point and will be computed 95% confidence intervals. Matched control patient samples (standard of care CD19.CAR-T in the absence of prophylaxis) will be available from a separate prospective biobanking protocol and will be utilized to compare to samples collected on this study protocol.

### 11.0 REFERENCES FOR EXAMPLE 1

1. Neelapu S S, Locke F L, Bartlett N L, et al. Axicabtagene Ciloleucel CAR T-Cell Therapy in Refractory Large B-Cell Lymphoma. New England Journal of Medicine. 2017; 377(26): 2531-2544.
2. Schuster S J, Bishop M R, Tam C S, et al. Tisagenlecleucel in Adult Relapsed or Refractory Diffuse Large B-Cell Lymphoma. New England Journal of Medicine. 2019; 380(1):45-56.
3. Lee D W, Santomasso B D, Locke F L, et al. ASTCT Consensus Grading for Cytokine Release Syndrome and Neurologic Toxicity Associated with Immune Effector Cells. Biology of Blood and Marrow Transplantation. 2019; 25(4): 625-638.
4. Norelli M, Camisa B, Barbiera G, et al. Monocyte-derived IL-1 and IL-6 are differentially required for cytokine-release syndrome and neurotoxicity due to CAR T cells. Nature Medicine. 2018; 24(6): 739-748.
5. Hunter C A, Jones S A. IL-6 as a keystone cytokine in health and disease. Nature Immunology. 2015; 16(5): 448-457.
6. Teachey D T, Lacey S F, Shaw P A, et al. Identification of Predictive Biomarkers for Cytokine Release Syndrome after Chimeric Antigen Receptor T-cell Therapy for Acute Lymphoblastic Leukemia. Cancer Discovery. 2016; 6(6): 664-679.
7. Maude S L, Frey N, Shaw P A, et al. Chimeric Antigen Receptor T Cells for Sustained Remissions in Leukemia. New England Journal of Medicine. 2014; 371(16): 1507-1517.
8. Davila M L, Riviere I, Wang X, et al. Efficacy and Toxicity Management of 19-28z CAR T Cell Therapy in B Cell Acute Lymphoblastic Leukemia. Science Translational Medicine. 2014; 6(224): 224ra225-224ra225.
9. Obstfeld A E, Frey N V, Mansfield K, et al. Cytokine release syndrome associated with chimeric-antigen receptor T-cell therapy: clinicopathological insights. Blood. 2017; 130(23):2569-2572.
10. Lee D W, Gardner R, Porter D L, et al. Current concepts in the diagnosis and management of cytokine release syndrome. Blood. 2014; 124(2): 188-195.
11. Chen F, Teachey D T, Pequignot E, et al. Measuring IL-6 and sIL-6R in serum from patients treated with tocilizumab and/or siltuximab following CAR T cell therapy. Journal of Immunological Methods. 2016; 434:1-8.
12. Nishimoto N, Terao K, Mima T, Nakahara H, Takagi N, Kakehi T. Mechanisms and pathologic significances in increase in serum interleukin-6 (IL-6) and soluble IL-6 receptor after administration of an anti-IL-6 receptor antibody, tocilizumab, in patients with rheumatoid arthritis and Castleman disease. Blood. 2008; 112(10): 3959-3964.
13. Kadauke S, Myers R M, Li Y, et al. Risk-Adapted Preemptive Tocilizumab to Prevent Severe Cytokine Release Syndrome After CTL019 for Pediatric B-Cell Acute Lymphoblastic Leukemia: A Prospective Clinical Trial. Journal of Clinical Oncology. 2021; 39(8):920-930.
14. Frederick L. Locke S S N, Nancy L. Bartlett, Lazaros J. Lekakis, Caron A. Jacobson, Ira Braunschweig, Olalekan O. Oluwole, Tanya Siddiqi, Yi Lin, John M Timmerman, Patrick M. Reagan, Adrian Bot, John M. Rossi, Marika Sherman, Lynn Navale, Yizhou Jiang, Jeff S. Aycock, Meg Elias, Jeffrey S. Wiezorek, William Y. Go, David B. Miklos. Preliminary Results of Prophylactic Tocilizumab after Axicabtageneciloleucel (axi-cel; KTE-C19) Treatment for Patients with Refractory, Aggressive Non-Hodgkin Lymphoma (NHL). Blood. 2017; 130:1547.
15. Caimi P F, Pacheco Sanchez G, Sharma A, et al. Prophylactic Tocilizumab Prior to Anti-CD19 CAR-T Cell Therapy for Non-Hodgkin Lymphoma. Front Immunol. 2021; 12:745320.
16. Van Rhee F, Wong R S, Munshi N, et al. Siltuximab for multicentric Castleman's disease: a randomised, double-blind, placebo-controlled trial. The Lancet Oncology. 2014; 15(9):966-974.
17. Oluwole O O, Bouabdallah K, Muñoz J, et al. Prophylactic corticosteroid use in patients receiving axicabtagene ciloleucel for large B-cell lymphoma. British Journal of Haematology. 2021; 194(4):690-700.
18. Mahmoudjafari Z, Hawks K G, Hsieh A A, Plesca D, Gatwood K S, Culos K A. American Society for Blood and Marrow Transplantation Pharmacy Special Interest Group Survey on Chimeric Antigen Receptor T Cell Therapy Administrative, Logistic, and Toxicity Management Practices in the United States. Biology of Blood and Marrow Transplantation. 2019; 25(1): 26-33.
19. Riegler L L, Jones G P, Lee D W. <p>Current approaches in the grading and management of cytokine release syndrome after chimeric antigen receptor T-cell therapy</p>. Therapeutics and Clinical Risk Management. 2019; Volume 15:323-335.
20. Kurzrock R, Voorhees P M, Casper C, et al. A Phase I, Open-Label Study of Siltuximab, an Anti-IL-6 Monoclonal Antibody, in Patients with B-cell Non-Hodgkin Lymphoma, Multiple Myeloma, or Castleman Disease. Clinical Cancer Research. 2013; 19(13): 3659-3670.
21. Schuster S J, Svoboda J, Chong E A, et al. Chimeric Antigen Receptor T Cells in Refractory B-Cell Lymphomas. New England Journal of Medicine. 2017; 377(26): 2545-2554.
22. Kochenderfer J N, Somerville R P T, Lu T, et al. Lymphoma Remissions Caused by Anti-CD19 Chimeric Antigen Receptor T Cells Are Associated With High Serum Interleukin-15 Levels. Journal of Clinical Oncology. 2017; 35(16):1803-1813.
23. Cheson B D, Fisher R I, Barrington S F, et al. Recommendations for Initial Evaluation, Staging, and Response Assessment of Hodgkin and Non-Hodgkin Lymphoma: The Lugano Classification. Journal of Clinical Oncology. 2014; 32(27): 3059-3067.

### LIST OF ABBREVIATIONS

ADCC Antibody dependent cellular cytotoxicity
ADCP Antibody dependent cellular phagocytosis
AE Adverse event
ALL Acute lymphoblastic leukemia
ALT Alanine aminotransferase
Alk Phos Alkaline phosphatase
AST Aspartate aminotransferase
BOR Best overall response
C Cycle
CAP College of American Pathologists
CDC Complement-dependent cytotoxicity
CLL Chronic Lymphocytic Leukemia
CML Chronic myeloid leukemia
CNS Central nervous system
CPO Clinical Protocol Office
CR Complete response
CRS Cytokine release syndrome
CT Computer tomography
CyTOF Mass cytometry
CD19.CAR-T CD19 directed chimeric antigen receptor T-cell therapy
D Day
DLBCL Diffuse large B-cell lymphoma
DNA Deoxyribonucleic acid
DSMC Data safety monitoring committee
ECL Electrochemiluminescence
ECOG Eastern Cooperative Oncology Group
eCRF Electronic case report form
Fab Fragment, antigen binding
FDA Food and Drug Administration
GCP Good Clinical Practice
HBs-Ag Hepatitis B surface antigen
HBc Hepatitis B core
HBV Hepatitis B virus
HCL Hairy cell leukemia
HCV Hepatitis C virus
HIPAA Health Insurance Portability and Accountability Act
HIV Human immunodeficiency virus
ICANS Immune effector cell associated neurotoxicity syndrome
IDS Investigational drug service
Ig Immunoglobulin
IHC Immunohistochemistry
IV Intravenous
kg kilogram
L Liter
LDH Lactate dehydrogenase
mcL microliter
mg milligram
MI Myocardial infarction
MRD Minimal residual disease
MRI Magnetic resonance Imaging
MTV Metabolic tumor volume
NCI-CTCAE National Cancer Institute-Common Terminology Criteria
AE for Adverse Events
NHL Non-Hodgkin lymphoma
NK Natural killer
ORR Overall response rate, objective response rate
OS Overall survival
PBMC Peripheral blood mononuclear cells
PCR Polymerase chain reaction
PD Progressive disease
PET Positron Emission Tomography
PFT Pulmonary function test
PI Principal investigator
PK Pharmacokinetic(s)
PLL Prolymphocytic leukemia
PR Partial response
PRC Protocol review committee
RIS Reduction immunosuppression
RNA Ribonucleic acid
R/R Relapsed/Refractory
SAE Serious adverse event
SAR Serious adverse reaction
s.c. Subcutaneous
SD Stable disease
SLL Small Lymphocytic Lymphoma
SOT Solid organ transplant
Th T helper
ULN Upper limit of normal

### 12.0 APPENDICES

### 12.1 Appendix A. Revised Response Criteria for Lymphoma²³

| Response and Site | PET-CT-Based Response | CT-Based Response |
|---|---|---|
| Complete | Complete metabolic response | Complete radiologic |
| | | response (all of the |
| | | following) |
| Lymph nodes and | Score 1, 2, or 3* with or without | Target nodes/nodal |
| extra-lymphatic | a residual mass 5 PS^{†} | masses must regress |
| sites | It is recognized that in | to ≤1.5 cm in LDi |
| | Waldeyer's ring or extranodal | No extralymphatic |
| | sites with high physiologic | sites of disease |
| | uptake or with activation within | |
| | spleen or marrow (e.g., with | |
| | chemotherapy or myeloid colony- | |
| | stimulating factors), uptake may | |
| | be greater than normal mediastinum | |
| | and/or liver. In this circumstance, | |
| | complete metabolic response may | |
| | be interred if uptake at sites | |
| | f initial involvement is no greater | |
| | than surrounding normal tissue even | |
| | if the tissue has high physiologic | |
| | uptake. | |
| Nonmeasured | Not applicable | Absent |
| lesion | | |
| Organ | Not applicable | Regress to normal |
| enlargement | | |
| New lesions | None | None |
| Bone marrow | No evidence of FDG-avid disease | Normal by morphology; |
| | in marrow | if indeterminate, |
| | | IHC negative |
| Partial | Partial metabolic response | Partial remission (all |
| Lymph nodes and | Score of 4 or 5^{†} with reduced | of the following) |
| extra-lymphatic | uptake compared with baseline | ≥50% decrease in SPD |
| sites | and residual mass(es) of any size; | of up to 6 target |
| | At interim, these findings indicate | measurable nodes and |
| | responding disease; | extranodal sites |
| | At end of treatment, these findings | When a lesion is too |
| | indicate residual disease | small to measure on CT, |
| | | assign 5 mm × 5 mm as |
| | | the default value When no |
| | | longer visible, 0 × 0 mm |
| | | For a node >5 mm × 5 mm, |
| | | but smaller than normal, use |
| | | actual measurement for |
| | | calculation |
| Nonmeasured | Not applicable | Absent/normal, regressed, |
| lesions | | but no increase |
| Organ | Not applicable | Spleen must have regressed |
| enlargement | | by >50% in length beyond |
| | | normal |
| New lesions | None | None |
| Bone marrow | Residual uptake higher than uptake | Not applicable |
| | in normal marrow but reduced | |
| | compared w/baseline (diffuse | |
| | uptake compatible w. reactive | |
| | changes from chemotherapy allowed). | |
| | If there are persistent focal changes | |
| | in the marrow in the context of a | |
| | nodal response, consideration should | |
| | be given to further evaluation w/MRI | |
| | or biopsy or an interval scan | |
| No response or | No metabolic response | Stable disease |
| stable disease | | |
| Target nodes/ | Score 4 or 5 with no significant | <50% decrease from baseline |
| nodal masses, | | in SPD of |
| extranodal | change in FDG uptake from baseline | up to 6 dominant, measurable |
| lesions | at interim or end of treatment | nodes and extranodal sites; |
| | | no criteria for progressive |
| | | disease are met |
| Non measure | Not applicable | No increase consistent with |
| lesions | | progression |
| Organ | Not applicable | No increase consistent with |
| enlargement | | progression |
| New lesions | None | None |
| Bone marrow | No change from baseline | Not applicable |
| Progressive | Progressive metabolic disease | Progressive disease requires |
| disease | | at least 1 of the following |
| Individual | Score 4 or 5 with an increase in | PPD progression: |
| target | intensity of uptake from | |
| nodes/nodal | baseline and/or | |
| masses | | |
| Extranodal lesions | New FDG-avid foci consistent with | An individual node/lesion |
| | lymphoma at interim or end-of- | must be abnormal with: |
| | treatment assessment | LDi >1.5 cm and |
| | | Increase by ≥50% from |
| | | PPD nadir and an increase |
| | | in LDi or SDi from nadir |
| | | 0.5 cm for lesions ≤2 cm |
| | | 1.0 cm for lesions >2 cm |
| | | In the setting of splenomegaly, |
| | | the splenic length must |
| | | increase by >50% of the |
| | | extent of its prior increase |
| | | beyond baseline (e.g., a 15-cm |
| | | spleen must increase to >16 cm). |
| | | If no prior splenomegaly, |
| | | must increase by at least 2 |
| | | cm from baseline. |
| | | New or recurrent splenomegaly |
| Non measured | None | New or clear progression of |
| lesions | | preexisting nonmeasured lesions |
| New lesions | New FDG-avid foci consistent with | Regrowth of previously resolved |
| | lymphoma rather than another | lesions |
| | etiology (e.g., infection, | A new node >1.5 cm in any axis |
| | inflammation). | A new extranodal site >1.0 |
| | | cm in any axis; |
| | If uncertain regarding etiology | if <1.0 cm in any axis, its |
| | of new lesions, biopsy or interval | presence must be unequivocal |
| | scan may be considered | and must be attributable |
| | | to lymphoma |
| | | Assessable disease of any size |
| | | unequivocally attributable |
| | | to lymphoma |
| Bone marrow | New or recurrent FDG-avid foci | New or recurrent |

Revised Response Criteria (Table Key)
Abbreviations: 5PS, 5-point scale, CT computed tomography; FDG, fluorodeoxyglucose; IHC, immunohistochemistry; LDi, longest transverse diameter of a lesion; MRI, magnetic resonance imaging; PET, positron emission tomography; PPD cross product of the LDi and perpendicular diameter; SDi, shortest axis perpendicular to the LDi; SPD, sum of the product of the perpendicular diameters for multiple lesions.
*A score of 3 in many subjects indicates a good prognosis with standard treatment, especially if at the time of an interim scan. However, in trials involving PET where de-escalation is investigated, it may be preferable to consider a score of 3 as inadequate response (to avoid undertreatment). Measured dominant lesions: Up to 6 of the largest dominant nodes, nodal masses, and extranodal lesions selected to be clearly measurable in two diameters. Nodes should preferably be from disparate regions of the body and should include, where applicable, mediastinal and retroperitoneal areas. Non-nodal lesions include those preferably be from disparate regions of the body and should include, where applicable, mediastinal and retroperitoneal areas. Non-nodal lesions include those in solid organs (e.g., liver, spleen, kidneys, lungs), GI involvement, cutaneous lesions, or those noted on palpation. Non-measurable lesions: Any disease not selected as measure, dominant disease and truly assessable disease should be considered not measured. These sites include any nodes, nodal masses, and extranodal sites not selected as dominant or measurable or that do not meet the requirements for measurability but are still considered abnormal, as well as truly assessable disease, which is any site of suspected disease that would be difficult to follow quantitatively with measurement, including pleural effusions, ascites, bone lesions, leptomeningeal disease, abdominal masses, and other lesions that cannot be confirmed and followed by imaging. In Waldeyer's ring or in extranodal sites (e.g., GI tract, liver, bone marrow), FDG uptake may be greater than in the mediastinum with complete metabolic response, but should be no higher than surrounding normal physiologic uptake (e.g., with marrow activation as a result of chemotherapy or myeloid growth factors).
^{†}PET 5 PS: 1, no uptake above background; 2 uptake ≤ mediastinum; 3, uptake > mediastinum but ≤ liver; 4, uptake moderately > liver; 5, uptake markedly higher than liver and/or new lesions; X, new areas of uptake unlikely to be related to lymphoma.
Reference: Cheson BD, et al. Recommendations for initial evaluation, staging, and response assessment of hodgkin and non-hodgkin lymphoma: the lugano classification. J Clin Oncol. 2014; 32:3059-3067.

### 12.2 Appendix B. ECOG and KPS Performance Status

| ECOG Performance Status Scale | | Karnofsky Performance Scale (KPS) | |
|---|---|---|---|
| Grade | Descriptions | Percent | Description |
| 0 | Normal activity. Fully active, able | 100 | Normal, no complaints, no |
| | to carry on all pre-disease | | evidence of disease. |
| | performance without restriction. | 90 | Able to carry on normal |
| | | | activity; minor signs or |
| | | | symptoms of disease. |
| 1 | Symptoms, but ambulatory. | 80 | Normal activity with effort; |
| | Restricted in physically strenuous | | some signs or symptoms |
| | activity, but ambulatory and able | | of disease. |
| | to carry out work of a light or | 70 | Cares for self, unable to |
| | sedentary nature (e.g., light | | carry on normal activity or |
| | housework, office work). | | to do active work. |
| 2 | In bed <50% of the time. | 60 | Requires occasional |
| | Ambulatory and capable of all | | assistance, but is able to |
| | self-care, but unable to carry out | | care for most of his/her |
| | | | needs. |
| | any work activities. Up and about | 50 | Requires considerable |
| | more than 50% of waking hours. | | assistance and frequent |
| | | | medical care. |
| 3 | In bed >50% of the time. | 40 | Disabled, requires special |
| | Capable of only limited self-care, | | care and assistance. |
| | confined to bed or chair more | 30 | Severely disabled, |
| | than 50% of waking hours. | | hospitalization indicated. |
| | | | Death not imminent. |
| 4 | 100% bedridden. Completely | 20 | Very sick, hospitalization |
| | disabled. Cannot carry on any | | indicated. Death not |
| | | | imminent. |
| | self-care. Totally confined to bed | 10 | Moribund, fatal processes |
| | or chair. | | progressing rapidly. |
| 5 | Dead. | 0 | Dead. |

### 12.3 Appendix C. CRS Grading³

ASTCT CRS Consensus Grading

| CRS | | | | |
|---|---|---|---|---|
| Parameter | Grade 1 | Grade 2 | Grade 3 | Grade 4 |
| Fever* | Temperature ≥38° C. | Temperature ≥38° C. | Temperature ≥38° C. | Temperature ≥38° C. |
| | | With | | |
| Hypotension | None | Not requiring | Requiring a vasopressor | Requiring multiple |
| | | vasopressors | with or without vasopressin | vasopressors (excluding |
| | | | | vasopressin) |
| | | And/or^{†} | | |
| Hypoxia | None | Requiring low-flow nasal | Requiring high-flow nasal | Requiring positive |
| | | cannula^{‡} or blow-by | cannula^{‡}, facemask, | pressure (eg, CPAP, |
| | | | nonrebreather mask, or Venturi | BiPAP, intubation and |
| | | | mask | mechanical ventilation) |

Organ toxicities associated with CRS may be graded according to CTCAE v5.0 but they do not influence CRS grading.
*Fever is defined as temperature ≥38° C. not attributable to any other cause. In patients who have CRS then receive antipyretic or anticytokine therapy such as tocilizumab or steroids, fever is no longer required to grade subsequent CRS severity. In this case, CRS grading is driven by hypotension and/or hypoxia.
^{†}CRS grade is determined by the more severe event: hypotension or hypoxia not attributable to any other cause. For example, a patient with temperature of 39.5° C., hypotension requiring 1 vasopressor, and hypoxia requiring low-flow nasal cannula is classified as grade 3 CRS.
^{‡}Low-flow nasal cannula is defined as oxygen delivered at ≤6 L/minute. Low flow also includes blow-by oxygen delivery, sometimes used in pediatrics. High-flow nasal cannula is defined as oxygen delivered at >6 L/minute.

### 12.4 Appendix D. ICANS Grading³

ASTCT ICANS Consensus Grading for Adults

| Neurotoxicity Domain | Grade 1 | Grade 2 | Grade 3 | Grade 4 |
|---|---|---|---|---|
| ICE score* | 7-9 | 3-6 | 0-2 | 0 (patient is unarousable |
| | | | | and unable to perform ICE) |
| Depressed level | Awakens | Awakens | Awakens only to | Patient is unarousable or |
| of consciousness^{†} | spontaneously | to voice | tactile stimulus | requires vigorous or repetitive |
| | | | | tactile stimuli to arouse. |
| | | | | Stupor or coma |
| Seizure | N/A | N/A | Any clinical seizure focal | Life-threatening prolonged |
| | | | or generalized that resolves | seizure (>5 min); or Repetitive |
| | | | rapidly or nonconvulsive | clinical or electrical seizures |
| | | | seizures on EEG that resolve | without return to baseline in |
| | | | with intervention | between |
| Motor findings^{‡} | N/A | N/A | N/A | Deep focal motor weakness such |
| | | | | as hemiparesis or paraparesis |
| Elevated ICP/ | N/A | N/A | Focal/local edema on | Diffuse cerebral edema on ; |
| cerebral edema | | | neuroimaging§ | neuroimaging decerebrate or |
| | | | | decorticate posturing; or |
| | | | | cranial nerve VI palsy; or |
| | | | | papilledema; or Cushing's triad |

ICANS grade is determined by the most severe event (ICE score, level of consciousness, seizure, motor findings, raised ICP/cerebral edema) not attributable to any other cause; for example, a patient with an ICE score of 3 who has a generalized seizure is classified as grade 3 ICANS.
N/A indicates not applicable.
*A patient with an ICE score of 0 may be classified as grade 3 ICANS if awake with global aphasia, but a patient with an ICE score of 0 may be classified as grade 4 ICANS if unarousable.
^{†}Depressed level of consciousness should be attributable to no other cause (eg, no sedating medication).
^{‡}Tremors and myoclonus associated with immune effector cell therapies may be graded according to CTCAE v5.0, but they do not influence ICANS grading.
§Intracranial hemorrhage with or without associated edema is not considered a neurotoxicity feature and is excluded from ICANS grading. It may be graded according to CTCAE v5.0.

### 12.5 Appendix E. Recommended CRS Treatment Strategy

| CRS Grade | Management |
|---|---|
| Grade 1 | Supportive Care |
| Symptoms require symptomatic treatment | |
| only (e.g., fever, nausea, fatigue, headache, | |
| myalgia, malaise). | |
| Grade 2 | Supportive Care |
| Symptoms require and respond to moderate | Administer Siltuximab 11 mg/kg over 1 hour |
| intervention. | If no improvement in CRS grade after 12 hours, |
| Oxygen requirement less than 40% | administer additional dose of Siltuximab |
| FiO2 or hypotension responsive to fluids | 11 mg/kg over 1 hour. |
| or low-dose of one vasopressor or Grade 2 | If CRS grade worsens, administer Tocilizumab |
| organ toxicity | 8 mg/kg and consider steroids per institutional |
| | standards. |
| Grade 3 | *** Consider need for steroid therapy. |
| Symptoms require and respond to aggressive | If no steroids are indicated |
| intervention. | Administer Siltuximab 11 mg/kg over 1 hour |
| Oxygen requirement greater than or equal to | If no improvement in CRS grade after 12 hours, |
| 40% FiO2 or hypotension requiring high-dose | administer additional dose of Siltuximab |
| or multiple vasopressors or Grade 3 organ | 11 mg/kg over 1 hour. |
| toxicity or Grade 4 transaminitis. | If CRS grade worsens, administer Tocilizumab |
| | 8 mg/kg and consider steroids per institutional |
| | standards. |
| | If steroids are indicated |
| | Administer Tocilizumab 8 mg/kg and steroids |
| | per institutional standards. |
| Grade 4 | *** Strongly consider leading with steroid |
| Life-threatening symptoms. | therapy. |
| Requirements for ventilator support, | If no steroids are indicated |
| continuous veno-venous hemodialysis | Administer Siltuximab 11 mg/kg over 1 hour |
| (CVVHD) or Grade 4 organ toxicity | If no improvement in CRS grade after 12 hours, |
| (excluding transaminitis). | administer additional dose of Siltuximab |
| | 11 mg/kg over 1 hour. |
| | If steroids are indicated |
| | Administer Tocilizumab 8 mg/kg and steroids |
| | per institutional standards. |

### 12.6 Appendix F. Recommended ICANS Treatment Strategy

| Grading | | |
|---|---|---|
| Assessment | Concurrent CRS | No Concurrent CRS |
| Grade 1 | Supportive Care | Supportive Care |
| | If persistent Grade 1 ICANS >12 hours, | If persistent Grade 1 ICANS >12 hours, |
| | administer Siltuximab 11 mg/kg over 1 hour | administer Siltuximab 11 mg/kg over 1 hour |
| | If no improvement in CRS or ICANS grade | If no improvement in ICANS grade after |
| | after 12 hours, administer additional dose | 12 hours, administer additional dose of |
| | of Siltuximab 11 mg/kg over 1 hour. | Siltuximab 11 mg/kg over 1 hour. |
| | If CRS and/or ICANS grade worsens, | If ICANS grade worsens, administer steroids |
| | administer tocilizumab 8 mg/kg (for CRS) | per institutional standards. |
| | and administer steroids (for ICANS) per | |
| | institutional standards. | |
| Grade 2 | Administer steroids per institutional | Administer steroids per institutional |
| | standards | standards |
| | Administer Siltuximab 11 mg/kg over 1 hour | |
| | If no improvement in CRS or ICANS grade | |
| | after 12 hours, administer additional dose | |
| | of Siltuximab 11 mg/kg over 1 hour. | |
| | If CRS and/or ICANS grade worsens, | |
| | administer tocilizumab 8 mg/kg (for CRS) | |
| | and administer steroids (for ICANS) per | |
| | institutional standards. | |
| Grade 3 | Administer steroids per institutional | Administer steroids per institutional |
| | standards | standards |
| | Administer Siltuximab 11 mg/kg over 1 hour | |
| | If no improvement in CRS or ICANS grade | |
| | after 12 hours, administer additional dose | |
| | of Siltuximab 11 mg/kg over 1 hour. | |
| | If CRS and/or ICANS grade worsens, | |
| | administer tocilizumab 8 mg/kg (for CRS) | |
| | and administer steroids (for ICANS) per | |
| | institutional standards. | |
| Grade 4 | Management per institutional standards | Administer steroids per institutional |
| | with steroids with or without Tocilizumab. | standards |
| | Siltuximab is not recommended. | |

### Example 2: Pilot Study of Siltuximab Cytokine Release Syndrome Prophylaxis Prior to Treatment with Epcoritamab, a CD3/CD20 Bispecific Antibody for Treatment of B Cell Lymphomas

Primary objective: To evaluate the feasibility and efficacy of prophylactic administration of siltuximab prior to infusion of the first dose of epcoritamab with the purpose of preventing all-grade CRS.

### Secondary Objectives:

To determine the safety profile of a single dose of siltuximab as CRS prophylaxis
To determine the incidence of grade grade≥2 CRS after siltuximab prophylaxis
To determine the incidence of all grade and grade≥2 ICANS after Siltuximab prophylaxis.
To describe the adverse events after siltuximab prophylaxis.
To describe the disease response rates (overall and complete response rates) to epcoritamab in patients treated with prophylactic siltuximab.
To describe the rates of hospitalization for all causes and for cytokine release syndrome
To describe the cytokine profile of subjects treated with prophylaxis, including plasma concentrations of IL-6, interleukin 1, interleukin 10, tumor necrosis factor, interferon gamma and others. Comparisons with samples from patients treated without prophylaxis may be considered.
To describe the characteristics of T cell populations at baseline, and after epcoritamab infusion, weekly through the first cycle and on the first day of each cycle thereafter until cycle 6, day 1 (i.e. baseline, cycle 1, day 1, 8, 15 and cycles 2-6, day 1).

### Design

The proposed design of this phase 1-2 study would be a single cohort of one dose level of siltuximab, given at a dose of 11 mg/kg as infusion 1 hour prior to infusion of epcoritamab on day 1.

Patients would be observed for one 28-day cycle of epcoritamab for adverse events, including incidence of CRS, ICANS. If there are any unresolved toxicities considered secondary to siltuximab, subjects will continue to be followed until resolution. Patients will continue to be followed to evaluate disease response until disease progression.

Considering a single dosing cohort, the inventors propose including 20 subjects, with interim safety evaluation done after the first 3 subjects are enrolled.

If there are any unresolved toxicities considered secondary to siltuximab, subjects will continue to be followed until resolution.

### Patient Population

### Inclusion Criteria

1. CD20 positive Non-Hodgkin Lymphoma
   a. DLBCL (including high grade B cell lymphoma and Follicular lymphoma grade 3B and transformed follicular lymphoma) treated with at least 2 prior systemic antineoplastic therapies, including at least 1 anti-CD20 monoclonal antibody-containing therapy.
   b. FL grade 1-3A previously treated with at least 2 lines of systemic antineoplastic therapies, including at least 1 anti-CD20 monoclonal antibody-containing therapy.
   c. Mantle cell lymphoma, treated at least with at least 2 lines of systemic antineoplastic therapies, including at least 1 anti-CD20 monoclonal antibody-containing therapy AND at least 1 BTK inhibitor.
   d. Marginal zone lymphoma treated with at least 1 line of systemic antineoplastic therapy containing anti-CD20 monoclonal antibody containing therapy.
   e. Small lymphocytic lymphoma/chronic lymphocytic leukemia, treated with at least 2 lines of systemic antineoplastic therapies, including at least 1 BTK inhibitor and at least 1 BCL2 inhibitor.
2. At least 1 risk factor for cytokine release syndrome, including:
   a. age≥65 years,
   b. elevated lactate dehydrogenase,
   c. white blood cell count pre-anti-CD20 treatment>4.5×10⁹ cells/L,
   d. Ann Arbor Stage III/IV,
   e. sum of the product of the perpendicular diameters at study entry≥3000 mm²,
   f. cardiac comorbidity,
   g. bone marrow infiltration,
   h. circulating lymphoma cells in peripheral blood
3. ECOG 0-2

### Exclusion Criteria

1. Primary mediastinal B cell lymphoma
2. Primary CNS tumor or known CNS involvement at screening
3. History of severe allergic or anaphylactic reactions to anti-CD20 monoclonal antibody therapy
4. Active bacterial, viral, fungal, mycobacterial, parasitic or other infection requiring systemic therapy with 2 weeks prior to first dose of study drug
   Sample size: 20 patients

### Methodology

With a total of 20 patients and using a single stage exact binomial design, the inventors can detect a reduction in the CRS rate to 0.34 with 80% power and a type I error rate of 0.068. At the end of the trial, the inventors would reject the null hypothesis if there were 8 or fewer all-grade CRS events out of the total of 20 patients.

### Rationale

T cells are a component of the adaptive immune system, as effectors of cell mediated immunity. T cells exert their cytotoxic and potentially anti-tumoral effects upon engagement of the T cell receptor by a cognate peptide antigen, which is presented in the context of a major histocompatibility (MHC) molecule. Over the last decade, the concept of tumor-targeted T cells has become reality with chimeric antigen receptor (CAR) T cells and bispecific antibodies showing significant activity against B cell malignancies¹⁻⁴. While CAR T cells are genetically modified to target tumor surface molecules and exert their effects in an MHC-independent fashion, bispecific antibodies and bispecific T cell engagers exert their cytotoxicity through the simultaneous binding of tumor-associated surface molecule and CD3 in T cells, which activates T cells on the proximity of the malignant cells⁵.

Treatment with tumor-redirected T cells is associated with well described acute adverse events, including cytokine release syndrome (CRS) and immune effector cell associated neurologic syndrome (ICANS). In larger CAR T studies reported in lymphoma, CRS and ICANS occurred in 58% and 21%, respectively of patients treated with tisagenlecleucel² and 93% and 64%, respectively of patients treated with axicabtagene ciloleucel¹. In a phase 1/2 study of epcoritamab, the observed incidence of CRS in dose escalation was 69%, with no grade 3 events⁴. In dose expansion, incidence of CRS was reported at 49.7%, with 2.5% of patients experiencing ASTCT grade 3 CRS. A dose step-up of epcoritamab is used to mitigate the risk of CRS, with a priming and intermediate dose given on days 1 and 8, respectively, prior to continuing with full dose on day 15 of the first cycle. Komanduri and colleagues have proposed and validated a prediction model for incidence of CRS grade≥2 in patients treated with the bispecific antibody glofitamab⁶⁷. The risk factors identified include age≥65 years, elevated lactate dehydrogenase, white blood cell count pre-anti-CD20 treatment>4.5×10⁹ cells/L, Ann Arbor Stage III/IV, sum of the product of the perpendicular diameters at study entry≥3000 mm², cardiac comorbidity, bone marrow infiltration, and circulating lymphoma cells in peripheral blood.

These acute complications of T cell redirection are the result of T cell expansion and activation^{8, 9} leading to a hyperinflammatory state, with activated T cells acting in conjunction with other compartments of the immune system, including monocytes and macrophages^{10, 11}. Several core cytokines present rapid elevation, including interleukin (IL)-6, IL-10, tumor necrosis factor alpha (TNFα) and interferon gamma (IFNγ)⁹, and these elevations lead in turn to further increase of other cytokines. The cytokine responses in CRS and ICANS have IL-6 as a central mediator 12.

Cytokine release syndrome can be successfully treated with blockade of interleukin 6, either with tocilizumab, a monoclonal antibody that binds the soluble and membrane-bound forms of the IL-6 receptor13, 14 or siltuximab, a monoclonal antibody directed against IL-6 15.

The inventors have investigated the use of tocilizumab as prophylactic agent for prevention of severe CRS in adult non-Hodgkin lymphoma patients treated with anti-CD19 CAR-T cells16, 17. Tocilizumab was administered 1 hour prior to CAR-T infusion. The inventors treated 22 patients with prophylactic tocilizumab and 8 subjects without tocilizumab. They did not observe statistically significant differences in the baseline characteristics of patients. Cytokine release syndrome of any grade was observed in 6/8 (75%) of pts without prophylactic tocilizumab vs. 10/22 (45%) in pts treated with prophylactic tocilizumab (p=0.23), whereas CRS grade>1 was observed in 5 pts (62.5%) without prophylactic tocilizumab and in 4 pts (18%) treated with prophylactic tocilizumab (p<0.01). Patients treated with prophylaxis had lower peak concentrations of C reactive protein (1.1 mg/dL vs. 15.4 mg/dl, p<0.001) and ferritin (525 vs. 3778 ng/dl, p<0.01). There were no differences in peak lymphocyte counts and no differences in response rates.

Kauer and colleagues reported the use of tocilizumab prophylaxis prior to treatment with bispecific antibody treatment targeting PSMA and CD3, and observed there was significant CRS attenuation with this strategy18. Moreover, in vitro and in vivo studies showed that dexamethasone affected T cell proliferation and cytotoxicity, but not IL-6 signaling blockade.

These results indicate that a prophylactic strategy can decrease the incidence of severe CRS when given prior to epcoritamab.

Tocilizumab binding to the IL-6 receptor effectively removes the clearance mechanisms for IL-6, and increased IL-6 has been reported in subjects treated with tocilizumab19. IL-6 has been implicated in CAR-T related neurotoxicity 20. Siltuximab, as discussed above, binds IL-6 and has been used in the treatment of CRS1521. Because it binds IL-6 directly, siltuximab can overcome the theoretical concerns regarding IL-6 and ICANS, as siltuximab-bound IL-6 cannot penetrate the blood-brain barrier.

Based on this rationale, the inventors propose a pilot study investigating the use of siltuximab prophylaxis for prevention of cytokine release syndrome prior to treatment with epcoritamab in patients with relapsed/refractory B cell non-Hodgkin's lymphoma with 1 or more risk factors for CRS≥2.

### Treatment Plan

The proposed design of this study would be a single cohort of one dose level of siltuximab, administered at a dose of 11 mg/kg as infusion, 1 hour prior to infusion of the first dose of epcoritamab (Cycle 1, day 1). Patients would be followed for the incidence of CRS and ICANS on the first 28-day cycle of treatment.

Patients would receive epcoritamab at standard doses according to the dose ramp up established on prior trials:
Cycle 1, day 1: priming dose (0.16 mg)
Cycle 1, day 8, intermediate dose (0.8 mg)
Cycle 1, day 15: full dose (48 mg)
Cycles 2 and 3, days 1, 8, 15, 22: full dose (48 mg)
Cycles 4-9, days 1 and 15: full dose (48 mg)
Cycles 10 and onwards: full dose (48 mg)

Patients would also receive prophylactic medications as previously prescribed in epcoritamab trials, including:
Dexamethasone, 12 mg orally, on cycle 1, days 1-4, 8-11, 15-18 and 22-25, as well as cycle 2, days 1-4.
Diphenhydramine 50 mg intravenous or orally on cycle 1, days 1, 8, 15, 22 and cycle 2 day 1.
Acetaminophen, 650 mg orally on cycle 1, days 1, 8, 15, 22 and cycle 2 day 1.

CRS and ICANS assessments would be conducted throughout cycle 1 and cycle 2.

Study Calendar

| | | Cycle 1 | | | | | Cycles 2-3 | | | | Cycles 4-9 | | Cycle 10+ | |
|---|---|---|---|---|---|---|---|---|---|---|---|---|---|---|
| | | Day | Day | Day | Day | Day | Day | Day | Day | Day | Day | Day | Day | |
| | Baseline | 1 | 8 | 15 | 16 | 22 | 1 | 8 | 15 | 22 | 1 | 15 | 1 | EOT |
| Inclusion/ | X | | | | | | | | | | | | | |
| exclusion | | | | | | | | | | | | | | |
| Informed | X | | | | | | | | | | | | | |
| consent | | | | | | | | | | | | | | |
| Medical | X | X | X | X | X | X | X | | | | X | | X | X |
| history | | | | | | | | | | | | | | |
| Siltuximab | | X | | | | | | | | | | | | |
| prophylaxis | | | | | | | | | | | | | | |
| Epcoritamab | | X | X | X | | X | X | X | X | X | X | X | X | |
| Disease status/ | X | | | | | | X³ | | | | X³ | | X³ | |
| response | | | | | | | | | | | | | | |
| assessment | | | | | | | | | | | | | | |
| ECOG PS | X | X | X | X | X | X | X | X | X | X | X | X | X | |
| Vital Signs | X | X | X | X | X | X | X | X | X | X | X | X | X | |
| Physical | X | X | X | X | X | X | X | X | | | X | | X | |
| Exam | | | | | | | | | | | | | | |
| Neurologic | X | X | X | X | X | X | X | | | | X | | X | |
| examination | | | | | | | | | | | | | | |
| CRS/ICANS | | X | X | X | X | X | X | X | X | X | X | X | X | |
| assessment | | | | | | | | | | | | | | |
| Concomitant | X | X | X | X | X | X | X | X | X | X | X | X | X | |
| Meds | | | | | | | | | | | | | | |
| AE query | X | X | X | X | X | X | X | X | X | X | X | X | X | |
| Laboratory | X | X | X | X | X | X | X | X | X | X | X | | X | |
| studies¹ | | | | | | | | | | | | | | |
| Correlative² | X | X | X | X | X | X | X | X | | | X | | X | |

¹Laboratory studies include CBC, comprehensive metabolic function, C - reactive protein, ferritin, fibrinogen
²Correlative studies to be stored for batched analysis after study completion, include cytokines (until cycle 3 day 1), immunophenotype (lymphoid and myeloid) (until cycle 10 day 1 and EOT), with additional samples for ctDNA and proteomics (samples collected Cycle 1, day 1, 8, 15, 22, cycle 2-6, day 1, then EOT)
³Disease response to be done prior to cycle 3 day 1, then prior to cycle 6 and 9 and then every 3 months afterwards.

### Statistical Design Summary:

The inventors wish to test the null hypothesis H0: p≥p0 versus the alternative hypothesis H1: p<p0, where p0=0.59 based on the results of the phase 1/2 trial of epcoritamab alone (Hutchins M, et al., Lancet 2021).

With a total of 20 patients and using a single stage exact binomial design, the inventors can detect a reduction in the CRS rate to 0.34 with 80% power and a type I error rate of 0.068. At the end of the trial, the inventors would reject the null hypothesis if there were 8 or fewer all-grade CRS events out of the total of 20 patients.

### Details:

This study will be a phase 1-2 trial of siltuximab in combination with epcoritamab, to establish the safety and evaluate preliminary efficacy of the combination to prevent the occurrence of all-grade CRS.

The goal is to use siltuximab to reduce the rate of CRS, which was 59% (grade 1-2 only) in the initial phase 1/2 trial of epcoritamab.

Our primary outcome is the rate of CRS. The unacceptable rate is p0=0.59. The inventors fix a=0.1 and examine a one-sided alternative hypothesis, H0: p≥p0 versus H1: p<p0. Then the inventors will vary p1 and compute the associated power. The inventors use an exact binomial test.

| p1 | Power |
|---|---|
| 0.49 | 0.35 |
| 0.39 | 0.70 |
| 0.29 | 0.93 |
| 0.19 | 0.99 |

Based on this, if the alternative rate of p1=0.29, the inventors would reject the null hypothesis if there were 8 or fewer CRS events out of a total of 20 patients. This test has an actual power of 0.93 and an actual type I error of 0.068.

### References for Example 2

1. Neelapu S S, Locke F L, Bartlett N L, et al.: Axicabtagene Ciloleucel CAR T-Cell Therapy in Refractory Large B-Cell Lymphoma. New Engl J Medicine 377:2531-2544, 2017
2. Schuster S J, Bishop M R, Tam C S, et al.: Tisagenlecleucel in Adult Relapsed or Refractory Diffuse Large B-Cell Lymphoma. New Engl J Med 380:45-56, 2019
3. Viardot A, Goebeler M-E, Hess G, et al.: Phase 2 study of the bispecific T-cell engager (BiTE) antibody blinatumomab in relapsed/refractory diffuse large B-cell lymphoma. Blood 127: 1410-1416, 2016
4. Hutchings M, Mous R, Clausen M R, et al.: Dose escalation of subcutaneous epcoritamab in patients with relapsed or refractory B-cell non-Hodgkin lymphoma: an open-label, phase 1/2 study. Lancet 398: 1157-1169, 2021
5. Sun L L, Ellerman D, Mathieu M, et al.: Anti-CD20/CD3 T cell-dependent bispecific antibody for the treatment of B cell malignancies. Sci Transl Med 7:287ra70, 2015
6. Komanduri K V, Belousov A, Dixon M, et al.: Risk of Cytokine Release Syndrome with Glofitamab Is Predicted By an Updated Model with a Potential Clinical Application. Blood 140:9493-9495, 2022
7. Komanduri K V, Belousov A, Byrtek M, et al.: Development of a Predictive Model for Cytokine Release Syndrome to Inform Risk Stratification and CRS Management Following Immunotherapy. Blood 138: 1459-1459, 2021
8. Lee D W, Gardner R, Porter D L, et al.: Current concepts in the diagnosis and management of cytokine release syndrome. Blood 124: 188-195, 2014
9. Singh A, Dees S, Grewal I S: Overcoming the challenges associated with CD3+ T-cell redirection in cancer. Brit J Cancer 124:1037-1048, 2021
10. Norelli M, Camisa B, Barbiera G, et al.: Monocyte-derived IL-1 and IL-6 are differentially required for cytokine-release syndrome and neurotoxicity due to CAR T cells. Nat Med 24:739-748, 2018
11. Khadka R H, Sakemura R, Kenderian S S, et al.: Management of cytokine release syndrome: an update on emerging antigen-specific T cell engaging immunotherapies. Immunotherapy 11:851-857, 2019
12. Hunter C A, Jones S A: IL-6 as a keystone cytokine in health and disease. Nat Immunol 16:448-457, 2015
13. Neelapu S S, Tummala S, Kebriaei P, et al.: Chimeric antigen receptor T-cell therapy—assessment and management of toxicities. Nat Rev Clin Oncol 15:47-62, 2018
14. Morris E C, Neelapu S S, Giavridis T, et al.: Cytokine release syndrome and associated neurotoxicity in cancer immunotherapy. Nat Rev Immunol 1-12, 2021
15. Patel S, Cenin D, Corrigan D, et al.: Siltuximab for First-Line Treatment of Cytokine Release Syndrome: A Response to the National Shortage of Tocilizumab [Internet]140:5073-5074, 2022 Available from: doi.org/10.1182/blood-2022-169809
16. Caimi P F, Sanchez G P, Sharma A, et al.: Prophylactic Tocilizumab Prior to Anti-CD19 CAR-T Cell Therapy for Non-Hodgkin Lymphoma. Front Immunol 12:745320, 2021
17. Caimi P F, Ahmed N, Rojas P, et al.: Prophylactic tocilizumab before CD3/4-1bb anti-CD19 car-T cell infusion decreases incidence of severe crs without increased risk of neurotoxicity [Internet]. Cytotherapy 22:S16-S17, 2020 Available from: doi.org/10.1016/j.jcyt.2020.03.483
18. Kauer J, Hörner S, Osburg L, et al.: Tocilizumab, but not dexamethasone, prevents CRS without affecting antitumor activity of bispecific antibodies. J Immunother Cancer 8:e000621, 2020
19. Chen F, Teachey D T, Pequignot E, et al.: Measuring IL-6 and sIL-6R in serum from patients treated with tocilizumab and/or siltuximab following CAR T cell therapy. J Immunol Methods 434:1-8, 2016
20. Gust J, Hay K A, Hanafi L-A, et al.: Endothelial Activation and Blood-Brain Barrier Disruption in Neurotoxicity after Adoptive Immunotherapy with CD19 CAR-T Cells. Cancer Discov 7:1404-1419, 2017
21. Mahmoudjafari Z, Hawks K G, Hsieh A A, et al.: American Society for Blood and Marrow Transplantation Pharmacy Special Interest Group Survey on Chimeric Antigen Receptor T Cell Therapy Administrative, Logistic, and Toxicity Management Practices in the United States. Biol Blood Marrow Tr 25:26-33, 2019

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

1. A method of prophylactic therapy of a patient who is at risk of the development of cytokine release syndrome (CRS) and/or immune effector cell-associated neurotoxicity syndrome (ICANS) due to a therapeutic intervention;
the method comprising administering an antibody or fragment which is capable of inhibiting human IL-6 to the patient in an antibody dosage regimen in conjunction with the therapeutic intervention;
wherein the antibody dosage regimen comprises administering a pre-emptive dose of the antibody or fragment before the patient is at risk of the development of CRS and/or ICANS; and
wherein the therapeutic intervention comprises administration of one or more doses of a therapeutic agent.

2. The method of claim 1, wherein the antibody or fragment is a chimeric, humanized or CDR grafted antibody or fragment thereof comprising a heavy chain variable region in which CDR1, CDR2 and CDR3 comprise the amino acid sequences of SEQ ID NO: 1, SEQ ID NO: 2 and SEQ ID NO: 3, respectively; and a light chain variable region in which CDR1, CDR2 and CDR3 comprise the amino acid sequences of SEQ ID NO: 4, SEQ ID NO: 5 and SEQ ID NO: 6, respectively; and a constant region derived from a human IgG antibody.

3. The method of claim 2, wherein the antibody is siltuximab.

4. The method of claim 1, wherein the therapeutic intervention comprises administration of one or more doses of an immunotherapy agent.

5. The method of claim 1, wherein the pre-emptive dose of the antibody or fragment is administered between 5 days before and up to 1 day after commencement of the administration of at least one of the one or more doses of the therapeutic agent, optionally the immunotherapy agent.

6. The method of claim 5, wherein the pre-emptive dose of the antibody or fragment is administered between 24 hours before and up to 24 hours after commencement of the administration of the at least one dose of the therapeutic agent, optionally between 2 hours before and at the same time as commencement of the administration of the at least one dose of the therapeutic agent.

7. The method of claim 1, wherein the antibody or fragment is administered by intravenous administration, optionally by infusion, optionally over the course of one hour.

8. The method of claim 7, wherein the pre-emptive dose of the antibody is 11±3 mg/kg patient body weight, optionally 11 mg/kg; or wherein the pre-emptive dose of the fragment is a dose having an equivalent antagonistic effect on human IL-6.

9. The method of claim 1, wherein administering the pre-emptive dose of the antibody or fragment reduces the risk that the patient will develop CRS and/or ICANS, such as ≥grade 2 CRS and/or ICANS, such as ≥grade 3 CRS and/or ICANS; and/or reduces the grade of CRS and/or ICANS that the patient is at risk of developing; and/or reduces the duration of CRS and/or ICANS that the patient is at risk of developing.

10. The method of claim 1, wherein the antibody or fragment thereof is the only active agent administered pre-emptively as prophylaxis for CRS and/or ICANS.

11. The method of claim 1 wherein the antibody dosage regimen comprises administering a first treatment dose of the antibody or fragment after the pre-emptive dose, if clinically indicated.

12. The method of claim 11 wherein the first treatment dose of the antibody or fragment is clinically indicated if the patient develops CRS, optionally if the patient develops ≥grade 1 CRS, optionally if the patient develops ≥grade 2 CRS.

13. The method of claim 11 wherein the first treatment dose of the antibody or fragment is clinically indicated if the patient develops ICANS, optionally if the patient develops ≥grade 1 ICANS, optionally if the patient develops ≥grade 1 ICANS lasting for more than 12 hours.

14. The method of claim 12, wherein the first treatment dose of the antibody or fragment is administered within an hour of diagnosis of CRS and/or ICANS.

15. The method of claim 13 wherein the first treatment dose of the antibody or fragment is administered within 24 hours of diagnosis of ICANS, and optionally after ICANS has not improved for at least 6 hours, such as 12 hours.

16. The method of claim 11, wherein administering the first treatment dose of the antibody or fragment reduces the grade and/or duration of CRS and/or ICANS of the patient, reduces treatment-related mortality at 30 days, improves overall survival following diagnosis of CRS and/or ICANS, reduces number of days of intensive care treatment following diagnosis of CRS and/or ICANS, and/or reduces number of days of inpatient hospital treatment following diagnosis of CRS and/or ICANS.

17. The method of claim 12, wherein the antibody dosage regimen comprises administering a second treatment dose of the antibody or fragment after the first treatment dose, if clinically indicated.

18. The method of claim 17, wherein the second treatment dose of the antibody or fragment is clinically indicated if CRS and/or ICANS remains at the same grade at 12 hours after the first treatment dose.

19. The method of claim 17, wherein the second treatment dose of the antibody or fragment is administered between 12 and 24 hours after the first treatment dose.

20. The method of claim 17, wherein administering the second treatment dose of the antibody or fragment reduces the grade and/or duration of CRS and/or ICANS of the patient, reduces treatment-related mortality at 30 days, improves overall survival following administering the first treatment dose, reduces number of days of intensive care treatment following administering the first treatment dose, and/or reduces number of days of inpatient hospital treatment following administering the first treatment dose.

21. The method of claim 12, wherein the or each treatment dose of the antibody is 11±3 mg/kg patient body weight, optionally 11 mg/kg; or wherein the or each treatment dose of the fragment is a dose having an equivalent antagonistic effect on human IL-6.

22. The method of claim 12, wherein the patient is administered a steroid for the treatment of CRS and/or ICANS.

23. The method of claim 1, wherein the therapeutic intervention is for the treatment of a cancer, optionally a blood cancer or a solid tumour, optionally non-hodgkin lymphoma, which is optionally CD20 positive.

24. The method of claim 1, wherein the therapeutic agent is an immunotherapy agent which is an immune effector cell (IEC) therapy agent or a T-cell engaging (TCE) therapy agent, optionally a chimeric antigen receptor (CAR) T cell therapy agent; optionally wherein the therapeutic agent is administered as a single dose.

25. The method of claim 24, wherein the CAR T cell is directed to an antigen selected from the group consisting of CD19, B cell maturation antigen (BCMA); CD20; CD22; CD30; CD138; CD123; NKG2DL; CD5; CD7; CD4; KISS1 R; CLDN6; MUC21; MUC16; SLC6A3; QRFPR; GPR119; UPK2; ADAM12; SLC45A3; MS4A12; ALPP; SLC2A14; GS1-259H13.2; ADGRG2; ECEL1; ERVFRD-1; CHRNA2; GP2; PSG9; IL13Ra2; TAG-72; ErbB2; HER2; B7H3; PD-L1; EPCAM; NKG2D; MESO; CD70; SenL-T7; and CD79b.

26. The method of claim 25, wherein the CD19 directed CAR T cell is selected from the group consisting of tisagenlecleucel, axicabtagene ciloleucel, brexucabtagene autoleucel and lisocabtagene maraleucel.

27. The method of claim 25, wherein the BCMA directed CAR T cell is selected from idecabtagene vicleucel or ciltacabtagene autoleucel.

28. The method of claim 1, wherein the therapeutic agent is an immunotherapy agent which is a bispecific antibody; optionally wherein the bispecific antibody is administered in one, two or three doses of a step-up dosing schedule.

29. The method of claim 28, wherein the bispecific antibody comprises a first binding specificity for a T cell antigen, and a second binding specificity for a cancer antigen, optionally a cancer antigen selected from the group consisting of CD19, BCMA, CD20, CD22, CD30, CD79, CD138, PD1, GP100, EpCAM, GPRC5D, CD123 and DLL3.

30. The method of claim 28, wherein the bispecific antibody is a full-size IgG-like asymmetric bispecific antibody, optionally selected from the group consisting of Triomab, CrossMab, Duobody and BEAT; or is a single-chain variable fragment (scFv) antibody, optionally selected from the group consisting of bispecific T-cell engager (BiTE), dual-affinity re-targeting protein (DART), Tandem diabody (TandAb) and Immunotherapy antibody (ITab).

31. The method of claim 29, wherein the bispecific antibody is selected from the group consisting of Blinatumomab, Cadonilimab, Mosunetuzumab, Glofitamab, Epcoritamab, Teclistamab, Elranatamab, Erfonrilimab, Tebotelimab, Catumaxomab, Odronextamab, Talquetamab, Flotetuzumab, AFM13, Tarlatamab, TNB-383B and REGN5458.

32. The method of claim 1, wherein the patient has at least 1 risk factor for CRS.


SEQUENCE LISTING (8 sequences)

SEQ ID NO: 1 - protein, 5 aa, Homo sapiens
SFAMS

SEQ ID NO: 2 - protein, 17 aa, Homo sapiens
EISSGGSYTYYPDTVTG

SEQ ID NO: 3 - protein, 10 aa, Homo sapiens
GLWGYYALDY

SEQ ID NO: 4 - protein, 10 aa, Homo sapiens
SASSSVSYMY

SEQ ID NO: 5 - protein, 7 aa, Homo sapiens
DTSNLAS

SEQ ID NO: 6 - protein, 9 aa, Homo sapiens
QQWSGYPYT

SEQ ID NO: 7 - protein, 449 aa, Homo sapiens
EVQLVESGGKLLKPGGSLKLSCAASGFTFSSFAMSWFRQSPEKRLEWVAEISSGGSYTYYPDTVTGRFTISRDNAKNTLYLEMSSLRSEDTAMYYCARGLWGYYALDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 213 aa, Homo sapiens
QIVLIQSPAIMSASPGEKVTMTCSASSSVSYMYWYQQKPGSSPRLLIYDTSNLASGVPVRFSGSGSGTSYSLTISRMEAEDAATYYCQQWSGYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC